(19) United States
(12) Patent Application Publication
(10) Pub. No.: US 2022/0235333 A1
(43) Pub. Date: Jul. 28, 2022

(54) Modified Phenylalanine Dehydrogenase

(71) Applicant: AJINOMOTO CO., INC., Tokyo (JP)

(72) Inventors: Moemi Tatsumi, Kanagawa (JP); Kazutoshi Takahashi, Kanagawa (JP); Uno Tagami, Kanagawa (JP); Hiroki Yamaguchi, Kanagawa (JP)

(73) Assignee: AJINOMOTO CO., INC., Tokyo (JP)

(21) Appl. No.: 17/714,537

(22) Filed: Apr. 6, 2022

Related U.S. Application Data
(63) Continuation of application No. PCT/JP2020/038341, filed on Oct. 9, 2020.

(30) Foreign Application Priority Data
Oct. 11, 2019 (JP) 2019-187753

(51) Int. Cl. C12N 9/06; C12N 15/10; C12N 15/63; C12N 5/10

(52) U.S. Cl. CPC C12N 9/0018; C12N 5/10; C12N 15/63; C12N 15/10

(57) ABSTRACT

The present invention provides a unit and a method useful for more precise phenylalanine measurement. More specifically, the present invention provides a modified phenylalanine dehydrogenase that includes a mutation of at least one amino acid residue so as to improve the characteristics (for example, substrate specificity, solubility, and phenylalanine dehydrogenase activity) of a phenylalanine dehydrogenase related to measurement of phenylalanine, a method for analyzing phenylalanine by measuring phenylalanine contained in a test sample using the modified phenylalanine dehydrogenase, and others.

This application is a Continuation of, and claims priority under 35 U.S.C. § 120 to, International Application No. PCT/JP2020/038341, filed Oct. 9, 2020, and claims priority therethrough under 35 U.S.C. § 119 to Japanese Patent Application No. 2019-187753, filed Oct. 11, 2019, the entireties of which are incorporated by reference herein. Also, the Sequence Listing filed electronically herewith is hereby incorporated by reference (File name: 2022-04-06T_US-636_Seq_List; File size: 75 KB; Date recorded: Apr. 6, 2022).

### GENERAL FIELD

The present invention relates to a modified phenylalanine dehydrogenase, a method for analyzing phenylalanine using the same, and more.

### BACKGROUND ART

The amino acid phenylalanine is a biomarker stored in large amounts in blood and/or urine of patients having phenylketonuria, which is a hereditary phenylalanine metabolic disorder disease, and measurement of phenylalanine is very important for clinical diagnosis. A diet low in phenylalanine is required for phenylketonuria patients, and measurement of a phenylalanine content in food is also important. As a method for measuring phenylalanine, an enzymatic method of measurement using a phenylalanine dehydrogenase derived from Thermoactinomyces intermedius (Patent Literature 1, for example) is known.

As a method for measuring phenylalanine, an enzymatic method of measurement using a phenylalanine dehydrogenase derived from Thermoactinomyces intermedius (Patent Literature 1, for example) is known. In addition, analyses of amino acid residues taking part in the activity and the like of phenylalanine dehydrogenases have been performed (Non Patent Literatures 1 to 4).

### BACKGROUND ART REFERENCES

Patent Literature

Patent Literature 1: Japanese Examined Patent Application Publication No. H07-114713

Non Patent Literature 1: J Biochem. 1994 December; 116(6): 1370-1376

Non Patent Literature 2: Biochemistry 2000, 39, 31, 9174-9187

Non Patent Literature 3: Journal of Biotechnology Volume 142, Issue 2, 15 Jun. 2009, Pages 127-134

Non Patent Literature 4: PNAS 2016 113 (47) E7383-E7389

### SUMMARY

In the phenylalanine measurement using the modified enzyme described herein, the characteristics of the enzyme related to the ability to measure phenylalanine such as, for example, substrate specificity, solubility, and enzyme activity, are desirably excellent. However, wild-type, or non-modified, phenylalanine dehydrogenase is reactive with tyrosine, for example, and thus has low substrate specificity and low accuracy of measurement of phenylalanine, which is problematic. In addition, wild-type phenylalanine dehydrogenase is poorly soluble in water and is thus in liquid suspension, which causes segregation of the enzyme by flocculation or the like. Therefore, the influence of this segregation on the accuracy of measurement of phenylalanine is of concern. Given these circumstances, for practical implementation of a more precise phenylalanine measurement, an enzyme having higher substrate specificity, solubility, and/or enzyme activity is in demand.

An aspect of the present invention is to provide a modified phenylalanine dehydrogenase suitable for practical implementation of more precise phenylalanine measurement.

The subject matter as described herein is a method of measuring a phenylalanine concentration by using an enzyme with improved characteristics related to measurement of phenylalanine, for example, substrate specificity, solubility, and enzyme activity, resulting in development of a phenylalanine dehydrogenase with these improved characteristics.

It is an aspect of the present invention to provide a modified phenylalanine dehydrogenase, comprising a mutation of at least one amino acid residue in a motif selected from the group consisting of: (1) GPALGGXRM (SEQ ID NO:3); (2) GRFXTGTDMGT (SEQ ID NO:4); (3) DF; (4) GXANN (SEQ ID NO:5); (5) RH; (6) VNXGGLIQV (SEQ ID NO:6), and (7) combinations thereof, wherein X indicates any amino acid, and wherein said modified phenylalanine dehydrogenase comprises at least one motif selected from the motifs (1) to (6); wherein said modified phenylalanine dehydrogenase has at least one characteristic selected from the group consisting of a substrate specificity, solubility, and phenylalanine dehydrogenase activity higher than a non-modified phenylalanine dehydrogenase.

It is a further aspect of the present invention to provide the modified phenylalanine dehydrogenase as described above, in which the mutation is a substitution selected from the group consisting of: (a) substitution of leucine in GPALGGXRM (SEQ ID NO:3); (b) substitution of threonine, the seventh amino acid from the left, in GRFXTGTDMGT (SEQ ID NO:4); (c) substitution of phenylalanine in DF; (d) substitution of asparagine, the fourth amino acid from the left, in GXANN (SEQ ID NO:5); (e) substitution of arginine in RH; (f) substitution of asparagine in VNXGGLIQV (SEQ ID NO:6); (g) substitution of leucine in VNXGGLIQV (SEQ ID NO:6); (h) substitution of glutamine in VNXGGLIQV (SEQ ID NO:6); (i) substitution of valine, the ninth amino acid from the left, in VNXGGLIQV (SEQ ID NO:6), and (j) combinations thereof.

It is a further aspect of the present invention to provide the modified phenylalanine dehydrogenase as described above, in which the mutation is a substitution selected from the group consisting of: (a) substitution of leucine in GPALGGXRM (SEQ ID NO:3) with tryptophane, phenylalanine, tyrosine, or methionine; (b) substitution of threonine, seventh amino acid from the left, in GRFXTGTDMGT (SEQ ID NO:4) with serine; (c) substitution of phenylalanine in DF with leucine or isoleucine; (d) substitution of asparagine, fourth amino acid from the left in GXANN (SEQ ID NO:5) with glycine, glutamine, threonine, lysine, proline, or serine; (e) substitution of arginine in RH with aspartic acid or glutamic acid; (f) substitution of asparagine in VNXGGLIQV (SEQ ID NO:6) with valine, aspartic acid, methionine, glutamine, proline, isoleucine, histidine, alanine, threonine, glycine, or cysteine; (g) substitution of leucine in VNXGGLIQV (SEQ ID NO:6) with phenylalanine, glutamine, histidine, asparagine, isoleucine, aspartic acid, glycine, glutamic acid, threonine, or serine; (h) substitution of glutamine in VNXGGLIQV (SEQ ID NO:6) with aspartic acid, glutamic acid, lysine, asparagine, serine, or arginine; and substitution of valine, ninth amino acid residue from the left, in VNXGGLIQV (SEQ ID NO:6) with tyrosine, tryptophane, glutamic acid, asparagine, threonine, isoleucine, lysine, glycine, serine, leucine, methionine, glutamine, phenylalanine, cysteine, or arginine, and (j) combinations thereof.

It is a further aspect of the present invention to provide the modified phenylalanine dehydrogenase as described above, in which the phenylalanine dehydrogenase comprises motifs (1) to (6) in numerical order.

It is a further aspect of the present invention to provide the modified phenylalanine dehydrogenase according to as described above, in which the phenylalanine dehydrogenase is derived from the genus Thermoactinomyces.

It is a further aspect of the present invention to provide the modified phenylalanine dehydrogenase according to as described above, in which the phenylalanine dehydrogenase comprises: (A) the amino acid sequence of SEQ ID NO:1; (B) an amino acid sequence comprising a substitution, deletion, insertion, or addition of one or several amino acid residues in the amino acid sequence of SEQ ID NO:1; or (C) an amino acid sequence having 90% or more identity to the amino acid sequence of SEQ ID NO:1.

It is a further aspect of the present invention to provide a modified phenylalanine dehydrogenase, comprising a mutation of an amino acid residue corresponding to an amino acid residue selected from the group consisting of: R2, R10, Y11, C19, L41, G42, G43, C44, A50, S51, M66, C70, F77, K90, Y112, T115, D116, F124, R129, L137, K139, S140, K144, T147, K173, C200, C210, K216, K220, Q222, N227, R228, C234, C240, R255, C256, L257, N264, R271, Q277, K278, R279, S280, C282, N290, G293, L294, Q296, V297, R326, K328, N329, N331, C335, R340, K347, K348, and combinations thereof; wherein said phenylalanine dehydrogenase comprises: (A) the amino acid sequence of SEQ ID NO:1, (B) an amino acid sequence comprising substitution, deletion, insertion, or addition of one or several amino acid residues in the amino acid sequence of SEQ ID NO:1, or (C) an amino acid sequence having 90% or more identity to the amino acid sequence of SEQ ID NO:1, wherein said modified phenylalanine dehydrogenase has phenylalanine dehydrogenase activity, and wherein said phenylalanine dehydrogenase has an improved characteristic selected from the group consisting of substrate specificity, solubility, phenylalanine dehydrogenase activity, and combinations thereof.

It is a further aspect of the present invention to provide the modified phenylalanine dehydrogenase as described above, which comprises a substitution of an amino acid residue corresponding to an amino acid residue selected from group consisting of: R2D, R2E, R10D, R10E, Y11E, Y11D, C19A, C19S, L41W, L41F, L41Y, L41M, G42A, G43A, C44A, C44S, A50D, A50E, S51D, S51E, M66I, M66L, M66V, C70A, C70S, F77L, F77I, F77R, K90E, Y112L, T115S, D116E, F124L, F124I, R129K, L137V, K139E, S140A, K144G, T147A, T147S, T147N, K173E, K173D, C200A, C200S, C210S, C210A, K216D, K216E, K220D, K220E, Q222E, Q222D, N227D, N227E, R228E, R228D, C234A, C234S, C240S, C240A, R255E, R255D, C256A, C256S, L257K, N264G, N264Q, N264T, N264K, N264P, N264S, R271D, R271E, Q277D, K278D, K278E, R279D, R279E, S280D, C282S, C282A, N290V, N290D, N290M, N290Q, N290P, N290I, N290H, N290A, N290T, N290G, N290C, G293A, L294F, L294Q, L294H, L294N, L294I, L294D, L294G, L294E, L294T, L294S, Q296D, Q296E, Q296K, Q296N, Q296S, Q296R, V297Y, V297W, V297E, V297N, V297T, V297I, V297K, V297G, V297S, V297L, V297M, V297Q, V297F, V297C, V297R, R326E, K328E, K328D, N329D, N331E, N331D, C335A, C335S, R340D, R340E, K347D, K348E, and combinations thereof.

It is a further aspect of the present invention to provide method for analyzing phenylalanine, the method comprising measuring phenylalanine contained in a test sample using the modified phenylalanine dehydrogenase as described above.

It is a further aspect of the present invention to provide the method as described above, comprising mixing the test sample with nicotinamide adenine dinucleotide (NAD⁺) and detecting NADH produced from NAD⁺ by the action of the modified phenylalanine dehydrogenase.

It is a further aspect of the present invention to provide a method for producing phenylpyruvate, the method comprising producing phenylpyruvate from phenylalanine using the modified phenylalanine dehydrogenase as described above.

It is a further aspect of the present invention to provide a polynucleotide encoding the modified phenylalanine dehydrogenase as described above.

It is a further aspect of the present invention to provide an expression vector comprising the polynucleotide as described above.

It is a further aspect of the present invention to provide a transformant comprising an expression unit of a polynucleotide encoding the modified phenylalanine dehydrogenase as described above.

It is a further aspect of the present invention to provide a method for producing a modified phenylalanine dehydrogenase, the method comprising producing a modified phenylalanine dehydrogenase comprising a mutation of at least one amino acid residue so as to improve a characteristic selected from the group consisting of substrate specificity, solubility, phenylalanine dehydrogenase activity, and combinations thereof, using the transformant as described above.

It is a further aspect of the present invention to provide a kit for analyzing phenylalanine, the kit comprising the modified phenylalanine dehydrogenase as described above.

It is a further aspect of the present invention to provide the kit for analyzing phenylalanine as described above further comprising at least one of a buffer solution or a buffer salt for reaction and nicotinamide adenine dinucleotide (NAD⁺).

It is a further aspect of the present invention to provide an enzyme sensor for analyzing phenylalanine, the enzyme sensor comprising (a) an electrode for detection and (b) the modified phenylalanine dehydrogenase according to [1] immobilized or disposed on the electrode for detection.

The modified phenylalanine dehydrogenase as described herein has improved substrate specificity and is thus useful for quick, high-precision, and highly sensitive measurement of phenylalanine and/or production of phenylpyruvate. The modified phenylalanine dehydrogenase has improved solubility, thus does not cause segregation of enzyme by flocculation or the like, and is useful for uniform and high-precision measurement of phenylalanine. Furthermore, the modified phenylalanine dehydrogenase has improved phenylalanine dehydrogenase activity and is thus useful for quick and highly sensitive measurement of phenylalanine and/or production of phenylpyruvate. The modified phenylalanine dehydrogenase is useful as a liquid reagent in particular. The method of analysis is useful for diagnosis of diseases such as phenylketonuria and measurement of a phenylalanine content in food, for example.

### BRIEF DESCRIPTION OF DRAWINGS

FIG. 1 is a diagram of alignment of amino acid sequences near motif (1) (GPALGGXRM (SEQ ID NO:3)) of wild-type phenylalanine dehydrogenases derived from respective species. The arrows indicate representative mutation sites (L41, G42, and G43) for designing a modified phenylalanine dehydrogenase.

FIG. 2 is a diagram of alignment of amino acid sequences near motif (2) (GRFXTGTDMGT (SEQ ID NO:4)) and motif (3) (DF) of wild-type phenylalanine dehydrogenases derived from respective species. The arrows indicate representative mutation sites (T115 and F124).

FIG. 3 is a diagram of alignment of amino acid sequences near motif (4) (GXANN (SEQ ID NO:5)), motif (5) (RH), and motif (6) (VNXGGLIQV (SEQ ID NO:6)) of wild-type phenylalanine dehydrogenases derived from respective species. The arrows indicate representative mutation sites (N264, R271, N290, G293, L294, Q296, and V297) for designing a modified phenylalanine dehydrogenase.

### DETAILED DESCRIPTION OF EXEMPLARY EMBODIMENTS

A modified phenylalanine dehydrogenase is provided herein. The modified phenylalanine dehydrogenase can include a mutation of at least one amino acid residue so as to improve one or more characteristics related to measurement of phenylalanine such as substrate specificity, solubility, and phenylalanine dehydrogenase activity (hereinafter, also referred to simply as “enzyme activity” or “activity”).

Examples of the mutation of the amino acid residue include substitution, deletion, addition, and insertion; substitution is a particular example.

The amino acid residue to be mutated can be L-alanine (A), L-asparagine (N), L-cysteine (C), L-glutamine (Q), L-isoleucine (I), L-leucine (L), L-methionine (M), L-phenylalanine (F), L-proline (P), L-serine (S), L-threonine (T), L-tryptophane (W), L-tyrosine (Y), L-valine (V), L-aspartic acid (D), L-glutamic acid (E), L-arginine (R), L-histidine (H), L-lysine (K), or glycine (G) as a natural L-α-amino acid. When the mutation is substitution, addition, or insertion, the amino acid to be substituted, added, or inserted is the same as the amino acid residue to be mutated described above. In the following, L and α may be omitted in the expression of the amino acid.

A phenylalanine dehydrogenase (may be expressed as PheDH) is an oxidoreductase catalyzing the following reaction (EC 1.4.1.20):

L-phenylalanine+NAD⁺+H₂O←→phenylpyruvate+NH₄⁺+NADH.

An enzyme derived from, that is, native to, any organism (for example, microorganisms such as bacteria, actinomycetes, and fungi, insects, fishes, animals, and plants) can be used as the phenylalanine dehydrogenase from which the modified phenylalanine dehydrogenase is derived; examples thereof include phenylalanine dehydrogenases derived from bacteria belonging to the genus Thermoactinomyces (Thermoactinomyces intermedius and Thermoactinomyces sp., for example), bacteria belonging to the genus Lihuaxuella (Lihuaxuella thermophile, for example), bacteria belonging to the genus Baia (Baia soyae, for example), bacteria belonging to the genus Caldalkalibacillus (Caldalkalibacillus thermarum, for example), bacteria belonging to the genus Bacillus (Bacillus badius, Bacillus sp., Bacillus halodurans, and Lysinibacillus sphaericus (also called Bacillus sphaericus), for example), bacteria belonging to the genus Fictibacillus (Fictibacillus nanhaiensis, for example), bacteria belonging to the genus Lysinibacillus (Lysinibacillus sphaericus (also called Bacillus sphaericus), for example), bacteria belonging to the genus Sporosarcina (Sporosarcina ureae, for example), bacteria belonging to the genus Rhodococcus (Rhodococcus sp., for example), and bacteria belonging to genera related thereto. Among the bacteria, bacteria belonging to the genus Thermoactinomyces are a particular example, and Thermoactinomyces intermedius is an even more particular example. Table 1 below lists examples of the wild-type phenylalanine dehydrogenase:

TABLE 1
Examples of wild-type phenylalanine dehydrogenase

| | | | Identity (%) |
|---|---|---|---|
| | | | by |
| | | | GENETYX |
| | | GenBank | (registered |
| Species origin | UniProt ID | Accession No. | trademark) ^{※1} |
| Thermoactinomyces | P22823 | P22823.1 | — |
| intermedius | | | |
| Lihuaxuella thermophile | A0A1H8C | SEM88729.1 | 73 |
| | 1K2 | | |
| Baia soyae | — | TCP62478.1 | 57 |
| Thermoactinomyces sp. DSM | A0A1K2A | SFX81991.1 | 57 |
| 45891 | 6F4 | | |
| Caldalkalibacillus thermarum | F5L9G2 | F5L9G2.1 | 57 |
| TA2.A1 | | | |
| Bacillus badius | Q59224 | Q59224.1 | 55 |
| Fictibacillus nanhaiensis | A0A1B1P | ANT47406.1 | 54 |
| | GG4 | | |
| Bacillus sp. UTB2301 | Q9AJQ0 | BAB40584.1 | 54 |
| Lysinibacillus sphaericus | P23307 | P23307.1 | 53 |
| (Bacillus sphaericus) | | | |
| Sporosarcina ureae | P97014 | P97014.1 | 52 |
| Bacillus halodurans | Q9KG94 | BAB03937.1 | 51 |
| Rhodococcus sp. | Q59771 | Q59771.2 | 31 |

^{※1} The identity of the amino acid sequence with respect to the wild-type phenylalanine dehydrogenase derived from Thermoactinomyces intermedius was calculated on “Gaps are NOT taken into account.”

Examples of the wild-type phenylalanine dehydrogenase include wild-type phenylalanine dehydrogenases having at least one, for example, one, two, three, four, five, or six of the following motifs labelled (1) to (6). The wild-type phenylalanine dehydrogenase may include a plurality of (for example, two, three, four, five, or six) of the motifs (1) to (6) in this order. The term “order” refers to the direction from the N-terminus toward the C-terminus in an amino acid sequence. The wild-type phenylalanine may include all of the motifs (1) to (6), and all the motifs (1) to (6) may be in this order. In the following, each of the motifs is indicated by a one-letter expression amino acid sequence, in which X indicates any amino acid, such as, any of the 20 kinds of amino acids that form proteins, that is, alanine (A), asparagine (N), cysteine (C), glutamine (Q), isoleucine (I), leucine (L), methionine (M), phenylalanine (F), proline (P), serine (S), threonine (T), tryptophane (W), tyrosine (Y), valine (V), aspartic acid (D), glutamic acid (E), arginine (R), histidine (H), lysine (K), and glycine (G)).

motif (1):
(SEQ ID NO: 3)
a GPALGGXRM motif;
motif (2):
(SEQ ID NO: 4)
a GRFXTGTDMGT motif;
motif (3):
a DF motif;
motif (4):
(SEQ ID NO: 5)
a GXANN motif;
motif (5):
an RH motif;
and
motif (6):
(SEQ ID NO: 6)
a VNXGGLIQV motif.

Motifs (1), (2), (4), and (6) include the following motifs (1a), (2a), (4a), and (6a) and (6b), respectively, which are motifs indicated by shorter amino acid sequences. Consequently, the wild-type phenylalanine dehydrogenase may, for example, be a wild-type phenylalanine dehydrogenase that includes at least one (for example, one, two, three, four, five, six, or seven) of the motifs (1a), (2a), (3), (4a), (5), (6a), and (6b), and may also include a plurality of (for example, two, three, four, five, six, or seven) the motifs (1a), (2a), (3), (4a), (5), (6a), and (6b) in this order, and may include the motifs (1a), (2a), (3), (4a), (5), (6a), and (6b), and may include all of the motifs (1a), (2a), (3), (4a), (5), (6a), and (6b) in this order.

motif (1a):
(SEQ ID NO: 7)
a GPALGG motif in motif (1);
motif (2a):
(SEQ ID NO: 8)
a TGTDMGT motif in motif (2);
motif (4a):
an ANN motif in motif (4);
motif (6a):
a VN motif in motif (6);
and
motif (6b):
(SEQ ID NO: 9)
a GGLIQV motif in motif (6).

The modified phenylalanine dehydrogenase may include a mutation of at least one amino acid residue in at least one of the motifs (1) to (6), for example, at least one amino acid residue in at least one of the motifs (1a), (2a), (3), (4a), (5), (6a), and (6b)) in a phenylalanine dehydrogenase described above, having phenylalanine dehydrogenase activity, and showing at least one of substrate specificity, solubility, and phenylalanine dehydrogenase activity, which is higher than a wild-type phenylalanine dehydrogenase.

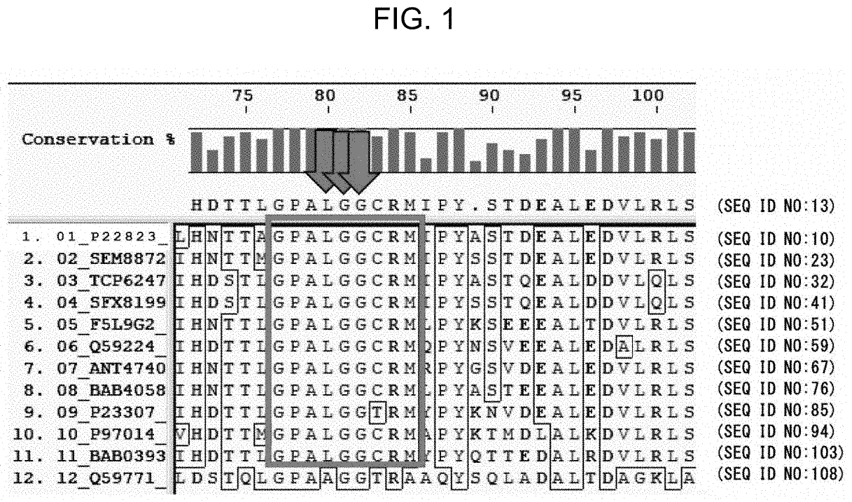

The mutation that imparts improved characteristics to the phenylalanine dehydrogenase related to an ability to measure phenylalanine is a substitution of leucine, the fifth amino acid glycine, or the sixth amino acid glycine (as counted from the left) in motif (1) (GPALGGXRM (SEQ ID NO:3)) of the amino acid sequence of the wild-type phenylalanine dehydrogenase. Motif (1) has nine consecutive amino acid residues of GPALGGXRM (SEQ ID NO:3) (X indicates any amino acid residue). The amino acid residue to be substituted can also be identified as leucine, the fifth amino acid glycine, or the sixth amino acid glycine in motif (1a) (GPALGG (SEQ ID NO:7)), indicated by the shorter amino acid sequence in motif (1). Although the position of motif (1) or (1a) in the amino acid sequence of the wild-type phenylalanine dehydrogenase can vary depending on the species origin of the enzyme, a person skilled in the art can determine the position of motif (1) or (1a) in the amino acid sequence of the wild-type phenylalanine dehydrogenase as appropriate and can thus identify the position of the leucine or the glycine (the fifth or sixth one) to be substituted. Normally, in the amino acid sequence of the phenylalanine dehydrogenase, motif (1) is located at positions 38 to 46, motif (1a) is located at positions 38 to 43, the leucine to be substituted is located at position 41, the glycine (the fifth one) to be substituted is located at position 42, and the glycine (the sixth one) to be substituted is located at position 43 (refer to, for example, Table 2 and FIG. 1).

TABLE 2
Sequences of conservation regions of motif (1) (GPALGGXRM (SEQ ID NO: 3)
motif) and motif (1a) (GPALGG (SEQ ID NO: 7) motif) in wild-type phenylalanine
dehydrogenases derived from respective species

| | | | | | Conservation region |
|---|---|---|---|---|---|
| | | | | Mutation | (underline indicates |
| | | GenBank | Identity | site | representative |
| | Species origin | Accession No. | (%) | conservation | mutation sites) |
| 1 | Thermoactinomyces | P22823.1 | —○ | ○ | |
| | intermedius | | | | |
| 2 | Lihuaxuella | SEM88729.1 | 73 | ○ | GPALGGCRMIPY |
| | thermophile | | | | (SEQ ID NO: 17) |
| 3 | Baia soyae | TCP62478.1 | 57 | ○ | GPALGGCRMIPY |
| | | | | | (SEQ ID NO: 27) |
| 4 | Thermoactinomyces sp. | SFX81991.1 | 57 | ○ | GPALGGCRMIPY |
| | DSM 45891 | | | | (SEQ ID NO: 36) |
| 5 | Caldalkalibacillus | F5L9G2.1 | 57 | ○ | GPALGGCRM |
| | thermarum TA2.A1 | | | | (SEQ ID NO: 45) |
| 6 | Bacillus badius | Q59224.1 | 55 | ○ | GPALGGCRM |
| | | | | | (SEQ ID NO: 55) |
| 7 | Fictibacillus | ANT47406.1 | 54 | ○ | GPALGGCRM |
| | nanhaiensis | | | | (SEQ ID NO: 63) |
| 8 | Bacillus sp. UTB2301 | BAB40584.1 | 54 | ○ | GPALGGCRM |
| | | | | | (SEQ ID NO: 71) |
| 9 | Lysinibacillus | P23307.1 | 53 | ○ | GPALGG |
| | sphaericus (Bacillus | | | | (SEQ ID NO: 80) |
| | sphaericus) | | | | |
| 10 | Sporosarcina ureae | P97014.1 | 52 | ○ | GPALGGCRM |
| | | | | | (SEQ ID NO: 89) |
| 11 | Bacillus halodurans | BAB03937.1 | 51 | ○ | GPALGGCRM |
| | | | | | (SEQ ID NO: 98) |
| 12 | Rhodococcus sp. | Q59771.2 | 31 | x | |

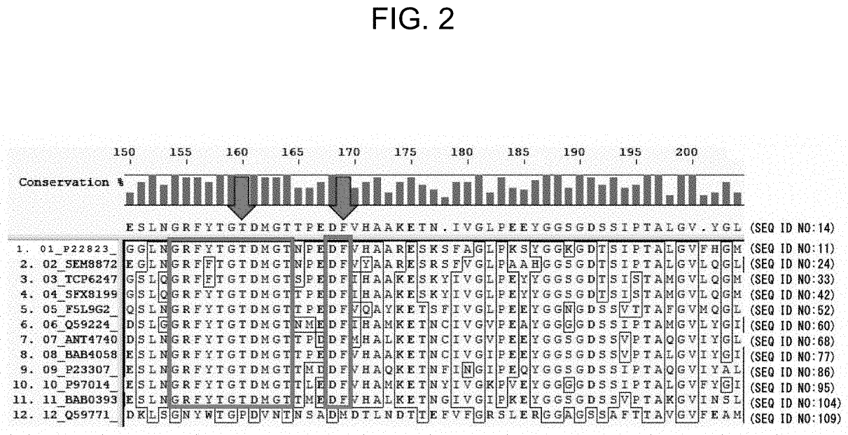

The mutation that imparts improved characteristics to the phenylalanine dehydrogenase related to an ability to measure phenylalanine is a substitution of the seventh amino acid threonine (as counted from the left) in motif (2) (GRFXTGTDMGT (SEQ ID NO:4)) of the amino acid sequence of the wild-type phenylalanine dehydrogenase. Motif (2) has 11 consecutive amino acid residues of GRFXTGTDMGT (SEQ ID NO:4) (X indicates any amino acid residue). The amino acid residue to be substituted can also be identified as the third amino acid threonine in motif (2a) (TGTDMGT (SEQ ID NO:8)) indicated by the shorter amino acid sequence in motif (2). Although the position of motif (2) or (2a) in the amino acid sequence of the wild-type phenylalanine dehydrogenase can vary depending on the species origin of the enzyme, a person skilled in the art can determine the position of motif (2) or (2a) in the amino acid sequence of the wild-type phenylalanine dehydrogenase as appropriate and can thus identify the position of the threonine to be substituted. Normally, in the amino acid sequence of the phenylalanine dehydrogenase, motif (2) is located at positions 109 to 119, motif (2a) is located at positions 113 to 119, and the threonine to be substituted is located at position 115 (for example, refer to Table 3 and FIG. 2).

TABLE 3
Sequences of conservation regions of motif (2) (GRFXTGTDMGT (SEQ ID NO: 4)
motif) and motif (2a) (TGTDMGT (SEQ ID NO: 8) motif) in wild-type phenylalanine
dehydrogenases derived from respective species

| | | | | | Conservation region |
|---|---|---|---|---|---|
| | | GenBank | | Mutation | (underline indicates |
| | | Accession | Identity | site con- | representative |
| | Species origin | No. | (%) | servation | mutation sites) |
| 1 | Thermoactinomyces | P22823.1 | — | ○ | |
| | intermedius | | | | |
| 2 | Lihuaxuella | SEM88729.1 | 73 | ○ | TGTDMGTNPEDFV |
| | thermophile | | | | (SEQ ID NO: 18) |
| 3 | Baia soyae | TCP62478.1 | 57 | ○ | TGTDMGT |
| | | | | | (SEQ ID NO: 28) |
| 4 | Thermoactinomyces | SFX81991.1 | 57 | ○ | GRFYTGTDMGT |
| | sp. DSM 45891 | | | | (SEQ ID NO: 37) |
| 5 | Caldalkalibacillus | F5L9G2.1 | 57 | ○ | LNGRFYTGTDMGT |
| | thermarum | | | | (SEQ ID NO: 46) |
| | TA2.A1 | | | | |
| 6 | Bacillus badius | Q59224.1 | 55 | ○ | GRFYTGTDMGTN |
| | | | | | (SEQ ID NO: 56) |
| 7 | Fictibacillus | ANT47406.1 | 54 | ○ | LNGRFYTGTDMGT |
| | nanhaiensis | | | | (SEQ ID NO: 64) |
| 8 | Bacillus sp. | BAB40584.1 | 54 | ○ | LNGRFYTGTDMGT |
| | UTB2301 | | | | (SEQ ID NO: 72) |
| 9 | Lysinibacillus | P23307.1 | 53 | ○ | LNGRFYTGTDMGT |
| | sphaericus | | | | (SEQ ID NO: 81) |
| | (Bacillus | | | | |
| | sphaericus) | | | | |
| 10 | Sporosarcina | P97014.1 | 52 | ○ | LNGRFYTGTDMGT |
| | ureae | | | | (SEQ ID NO: 90) |
| 11 | Bacillus | BAB03937.1 | 51 | ○ | LNGRFYTGTDMGT |
| | halodurans | | | | (SEQ ID NO: 99) |
| 12 | Rhodococcus sp. | Q59771.2 | 31 | ○ | |

The mutation that imparts improved characteristics to the phenylalanine dehydrogenase related to an ability to measure phenylalanine is a substitution of phenylalanine in motif (3) (DF) of the amino acid sequence of the wild-type phenylalanine dehydrogenase. Motif (3) has two consecutive amino acid residues of DF. Although the position of motif (3) in the amino acid sequence of the wild-type phenylalanine dehydrogenase can vary depending on the species origin of the enzyme, a person skilled in the art can determine the position of motif (3) in the amino acid sequence of the wild-type phenylalanine dehydrogenase as appropriate and can thus identify the position of the phenylalanine to be substituted. Normally, in the amino acid sequence of the phenylalanine dehydrogenase, motif (3) is located at positions 123 to 124, and the phenylalanine to be substituted is located at position 124 (for example, refer to Table 4 and FIG. 2).

TABLE 4
Sequences of conservation regions of motif (3) (DF motif) in wild-type
phenylalanine dehydrogenases derived from respective species

| | | | | | Conservation region |
|---|---|---|---|---|---|
| | | GenBank | | Mutation | (underline indicates |
| | | Accession | Identity | site con- | representative |
| | Species origin | No. | (%) | servation | mutation sites) |
| 1 | Thermoactinomyces | P22823.1 | — | ○ | |
| | intermedius | | | | |
| 2 | Lihuaxuella | SEM88729.1 | 73 | ○ | TGTDMGTNPEDFV |
| | thermophile | | | | (SEQ ID NO: 19) |
| 3 | Baia soyae | TCP62478.1 | 57 | ○ | PEDF |
| | | | | | (SEQ ID NO: 29) |
| 4 | Thermoactinomyces | SFX81991.1 | 57 | ○ | PEDF |
| | sp. DSM 45891 | | | | (SEQ ID NO: 38) |
| 5 | Caldalkalibacillus | F5L9G2.1 | 57 | ○ | PEDFV |
| | thermarum TA2.A1 | | | | (SEQ ID NO: 47) |
| 6 | Bacillus badius | Q59224.1 | 55 | ○ | EDF |
| 7 | Fictibacillus | ANT47406.1 | 54 | ○ | DF |
| | nanhaiensis | | | | |
| 8 | Bacillus sp. | BAB40584.1 | 54 | ○ | PEDFVHAA |
| | UTB2301 | | | | (SEQ ID NO: 73) |
| 9 | Lysinibacillus | P23307.1 | 53 | ○ | DFVHA |
| | sphaericus | | | | (SEQ ID NO: 82) |
| | (Bacillus | | | | |
| | sphaericus) | | | | |
| 10 | Sporosarcina ureae | P97014.1 | 52 | ○ | EDFVHA |
| | | | | | (SEQ ID NO: 91) |
| 11 | Bacillus halodurans | BAB03937.1 | 51 | ○ | EDFVHA |
| | | | | | (SEQ ID NO: 100) |
| 12 | Rhodococcus sp. | Q59771.2 | 31 | x | |

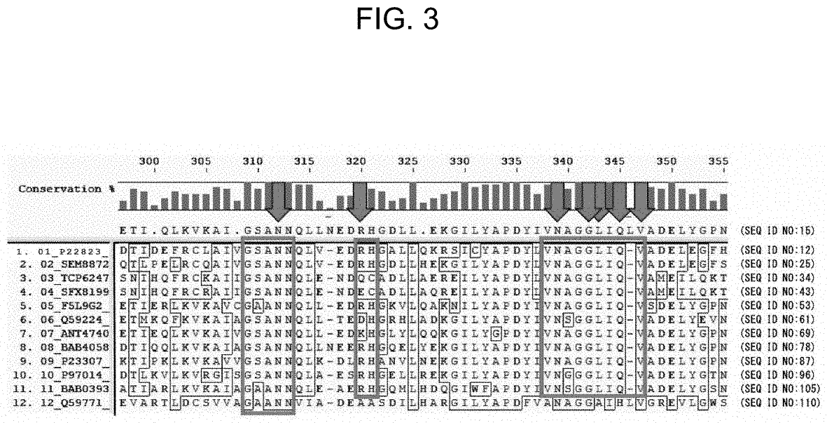

The mutation that imparts improved characteristics to the phenylalanine dehydrogenase related to an ability to measure phenylalanine is a substitution of asparagine as the fourth amino acid residue in motif (4) (GXANN (SEQ ID NO:5)) of the amino acid sequence of the wild-type phenylalanine dehydrogenase. Motif (4) has five consecutive amino acid residues of GXANN (SEQ ID NO:5) (X indicates any amino acid residue). The amino acid residue to be substituted can also be identified as asparagine as the second amino acid residue in motif (4a) (the ANN motif) indicated by the shorter amino acid sequence in motif (4). Although the position of motif (4) or (4a) in the amino acid sequence of the wild-type phenylalanine dehydrogenase can vary depending on the species origin of the enzyme, a person skilled in the art can determine the position of motif (4) or (4a) in the amino acid sequence of the wild-type phenylalanine dehydrogenase as appropriate and can thus identify the position of the asparagine to be substituted. Normally, in the amino acid sequence of the phenylalanine dehydrogenase, motif (4) is located at positions 261 to 265, motif (4a) is located at positions 263 to 265, and the asparagine to be substituted is located at position 264 (for example, refer to Table 5 and FIG. 3).

TABLE 5
Sequences of conservation regions of motif (4) (GXANN (SEQ ID NO: 5) motif) and
motif (4a) (ANN motif) in wild-type phenylalanine dehydrogenases derived from
respective species

| | | | | | Conservation region |
|---|---|---|---|---|---|
| | | GenBank | | Mutation | (underline indicates |
| | | Accession | Identity | site con- | representative |
| | Species origin | No. | (%) | servation | mutation sites) |
| 1 | Thermoactinomyces | P22823.1 | - | ○ | |
| | intermedius | | | | |
| 2 | Lihuaxuella | SEM88729.1 | 73 | ○ | AIVGSANNQLVED |
| | thermophile | | | | RHG |
| | | | | | (SEQ ID NO: 20) |
| 3 | Baia soyae | TCP62478.1 | 57 | ○ | GSANNQL |
| | | | | | (SEQ ID NO: 30) |
| 4 | Thermoactinomyces sp. | SFX81991.1 | 57 | ○ | GSANNQL |
| | DSM 45891 | | | | (SEQ ID NO: 39) |
| 5 | Caldalkalibacillus | F5L9G2.1 | 57 | ○ | ANNQL |
| | thermarum TA2.A1 | | | | (SEQ ID NO: 48) |
| 6 | Bacillus badius | Q59224.1 | 55 | ○ | GSANNQL |
| | | | | | ( [symbol] 57) |
| 7 | Fictibacillus | ANT47406.1 | 54 | ○ | AIVGSANNQL |
| | nanhaiensis | | | | (SEQ ID NO: 65) |
| 8 | Bacillus sp. UTB2301 | BAB40584.1 | 54 | ○ | GSANNQL |
| | | | | | (SEQ ID NO: 74) |
| 9 | Lysinibacillus | P23307.1 | 53 | ○ | VGSANNQL |
| | sphaericus (Bacillus | | | | (SEQ ID NO: 83) |
| | sphaericus) | | | | |
| 10 | Sporosarcina ureae | P97014.1 | 52 | ○ | GSANNQL |
| | | | | | (SEQ ID NO: 92) |
| 11 | Bacillus halodurans | BAB03937.1 | 51 | ○ | ANNQL |
| | | | | | (SEQ ID NO: 101) |
| 12 | Rhodococcus sp. | Q59771.2 | 31 | ○ | ANN |

The mutation that imparts improved characteristics to the phenylalanine dehydrogenase related to an ability to measure phenylalanine is a substitution of arginine in motif (5) (RH) of the amino acid sequence of the wild-type phenylalanine dehydrogenase. Motif (5) has two consecutive amino acid residues of RH. Although the position of motif (5) in the amino acid sequence of the wild-type phenylalanine dehydrogenase can vary depending on the derivation of the enzyme, a person skilled in the art can determine the position of motif (5) in the amino acid sequence of the wild-type phenylalanine dehydrogenase as appropriate and can thus identify the position of the arginine to be substituted. Normally, in the amino acid sequence of the phenylalanine dehydrogenase, motif (5) is located at positions 271 to 272, and the arginine to be substituted is located at position 271 (for example, refer to Table 6 and FIG. 3).

TABLE 6
Sequences of conservation regions of motif (5) (RH motif) in wild-type
phenylalanine dehydrogenases derived from respective species

| | | | | | Conservation region |
|---|---|---|---|---|---|
| | | GenBank | | Mutation | (underline indicates |
| | | Accession | Identity | site con- | representative |
| | Species origin | No. | (%) | servation | mutation sites) |
| 1 | Thermoactinomyces | P22823.1 | - | ○ | |
| | intermedius | | | | |
| 2 | Lihuaxuella | SEM88729.1 | 73 | ○ | AIVGSANNQLVEDR |
| | thermophile | | | | HG |
| | | | | | (SEQ ID NO: 21) |
| 3 | Baia soyae | TCP62478.1 | 57 | x | |
| 4 | Thermoactinomyces | SFX81991.1 | 57 | x | |
| | sp. DSM 45891 | | | | |
| 5 | Caldalkalibacillus | F5L9G2.1 | 57 | ○ | EDRHG |
| | thermarum TA2.A1 | | | | (SEQ ID NO: 49) |
| 6 | Bacillus badius | Q59224.1 | 55 | x | |
| 7 | Fictibacillus | ANT47406.1 | 54 | x | |
| | nanhaiensis | | | | |
| 8 | Bacillus sp. | BAB40584.1 | 54 | ○ | RHG |
| | UTB2301 | | | | |
| 9 | Lysinibacillus | P23307.1 | 53 | ○ | RH |
| | sphaericus (Bacillus | | | | |
| | sphaericus) | | | | |
| 10 | Sporosarcina ureae | P97014.1 | 52 | ○ | RHG |
| 11 | Bacillus halodurans | BAB03937.1 | 51 | ○ | RHG |
| 12 | Rhodococcus sp. | Q59771.2 | 31 | x | |

The mutation that imparts improved characteristics to the phenylalanine dehydrogenase related to an ability to measure phenylalanine is a substitution of asparagine, the fifth amino acid glycine, leucine, glutamine, or the ninth amino acid valine in motif (6) (the VNXGGLIQV (SEQ ID NO:6) motif) of the amino acid sequence of the wild-type phenylalanine dehydrogenase. Motif (6) has nine consecutive amino acid residues of VNXGGLIQV (SEQ ID NO:6) (X indicates any amino acid residue). The amino acid residue to be substituted can also be identified as asparagine in motif (6a) (VN) indicated by the shorter amino acid sequence in motif (6) or the second amino acid glycine, leucine, glutamine, or valine in motif (6b) (GGLIQV (SEQ ID NO:9)) indicated by the shorter amino acid sequence in motif (6). Although the position of motif (6), (6a), or (6b) in the amino acid sequence of the wild-type phenylalanine dehydrogenase can vary depending on the derivation of the enzyme, a person skilled in the art can determine the position of motif (6), (6a), or (6b) in the amino acid sequence of the wild-type phenylalanine dehydrogenase as appropriate and can thus identify the position of the asparagine, the glycine, the leucine, the glutamine, or the valine to be substituted. Normally, in the amino acid sequence of the phenylalanine dehydrogenase, motif (6) is located at positions 289 to 297, motif (6a) is located at positions 289 to 290, motif (6b) is located at positions 292 to 297, the asparagine to be substituted is located at position 290, the glycine to be substituted is located at position 293, the leucine to be substituted is located at position 294, the glutamine to be substituted is located at position 296, and the valine to be substituted is located at position 297, (for example, refer to Table 7 and FIG. 3).

TABLE 7
Sequences of conservation regions of motif (6) (VNXGGLIQV (SEQ ID NO: 6)
motif), motif (6a) (VN motif), and motif (6b) (GGLIQV (SEQ ID NO: 9) motif)
in wild-type phenylalanine dehydrogenases derived from respective species

| | | | | | Conservation region |
|---|---|---|---|---|---|
| | | GenBank | | Mutation | (underline indicates |
| | | Accession | Identity | site con- | representative |
| | Species origin | No. | (%) | servation | mutation sites) |
| 1 | Thermoactinomyces | P22823.1 | — | ○ | |
| | intermedius | | | | |
| 2 | Lihuaxuella | SEM88729.1 | 73 | ○ | YAPDYLVNAGGLIQ |
| | thermophile | | | | VADELEGF |
| | | | | | (SEQ ID NO: 22) |
| 3 | Baia soyae | TCP62478.1 | 57 | ○ | YAPDYLVNAGGLIQ |
| | | | | | VA |
| | | | | | (SEQ ID NO: 31) |
| 4 | Thermoactinomyces | SFX81991.1 | 57 | ○ | YAPDYLVNAGGLIQ |
| | sp. DSM 45891 | | | | VA |
| | | | | | (SEQ ID NO: 40) |
| 5 | Caldalkalibacillus | F5L9G2.1 | 57 | ○ | VNAGGLIQV |
| | thermarum TA2.A1 | | | | (SEQ ID NO: 50) |
| 6 | Bacillus badius | Q59224.1 | 55 | ○ | VNGGLIQVADEL |
| | | | | | (SEQ ID NO: 58) |
| 7 | Fictibacillus | ANT47406.1 | 54 | ○ | VNAGGLIQVADEL |
| | nanhaiensis | | | | (SEQ ID NO: 66) |
| 8 | Bacillus sp. | BAB40584.1 | 54 | ○ | VNAGGLIQVADEL |
| | UTB2301 | | | | (SEQ ID NO: 75) |
| 9 | Lysinibacillus | P23307.1 | 53 | ○ | VNAGGLIQVADEL |
| | sphaericus (Bacillus | | | | (SEQ ID NO: 84) |
| | sphaericus) | | | | |
| 10 | Sporosarcina ureae | P97014.1 | 52 | ○ | VNGGLIQVADEL |
| | | | | | (SEQ ID NO: 93) |
| 11 | Bacillus halodurans | BAB03937.1 | 51 | ○ | VNGGLIQVADEL |
| | | | | | (SEQ ID NO:102) |
| 12 | Rhodococcus sp. | Q59771.2 | 31 | ○ | NAGG |
| | | | | | (SEQ ID NO: 107) |

The modified phenylalanine dehydrogenase can be produced by introducing a mutation to a wild-type enzyme having one or more of the motifs (1) to (6). The wild-type enzyme may have two motifs, have three motifs, have four motifs, have five motifs, or six motifs selected from motifs (1) to (6). The modified phenylalanine dehydrogenase can be produced by introducing a mutation to a wild-type enzyme having one or more of the motifs (1a), (2a), (3), (4a), (5), (6a), and (6b). The wild-type enzyme may have two motifs, have three motifs, have four motifs, have five motifs, have six motifs, or have seven motifs selected from motifs (1a), (2a), (3), (4a), (5), (6a), and (6b).

Examples of the characteristics of the phenylalanine dehydrogenase related to measurement of phenylalanine include substrate specificity, solubility, and enzyme activity. The modified phenylalanine dehydrogenase may have only one of the characteristics described above or have two or three characteristics of the characteristics described above in combination.

The amino acid residues identified by motifs (1) to (6) may be identified by motifs (1a), (2a), (3), (4a), (5), (6a), and (6b) based on the corresponding relations described above.

As to the mutation in the six motifs described above, examples of the mutation (a single mutation or a combination with another mutation) improving at least one characteristic selected from substrate specificity, solubility, and enzyme activity include the following:

(a) a substitution of leucine in motif (1) with tryptophane, phenylalanine, tyrosine, or methionine;

(a+) a substitution of glycine as the fifth amino acid residue in motif (1) with alanine;

(a++) a substitution of glycine as the sixth amino acid residue in motif (1) with alanine;

(b) a substitution of threonine as the seventh amino acid residue in motif (2) with serine;

(c) a substitution of phenylalanine in motif (3) with leucine or isoleucine;

(d) a substitution of asparagine as the fourth amino acid residue in motif (4) with glycine, glutamine, threonine, lysine, proline, or serine;

(e) a substitution of arginine in motif (5) with aspartic acid or glutamic acid;

(f) a substitution of asparagine in motif (6) with valine, aspartic acid, methionine, glutamine, proline, isoleucine, histidine, alanine, threonine, glycine, or cysteine;

(g-) a substitution of glycine as the fifth amino acid residue in motif (6) with alanine;

(g) a substitution of leucine in motif (6) with phenylalanine, glutamine, histidine, asparagine, isoleucine, aspartic acid, glycine, glutamic acid, threonine, or serine;

(h) a substitution of glutamine in motif (6) with aspartic acid, glutamic acid, lysine, asparagine, serine or arginine; and

(i) a substitution of valine as the ninth amino acid residue in motif (6) with tyrosine, tryptophane, glutamic acid, asparagine, threonine, isoleucine, lysine, glycine, serine, leucine, methionine, glutamine, phenylalanine, cysteine, or arginine.

When the modified phenylalanine dehydrogenase has two or more amino acid residues of the phenylalanine dehydrogenase mutated, at least one or at least two of the mutations of the amino acid residues may be selected from the mutation of the amino acid residue described above. Furthermore, all the mutations of the amino acid residues may be selected from the mutation of the amino acid residue described above.

When the modified phenylalanine dehydrogenase includes two or more mutations, one or more of the following mutations can be included:

(a) a substitution of leucine in motif (1);

(b) a substitution of threonine as the seventh amino acid residue in motif (2);

(c) a substitution of phenylalanine in motif (3);

(e) a substitution of arginine in motif (5);

(f) a substitution of asparagine in motif (6);

(g) a substitution of leucine in motif (6);

(h) a substitution of glutamine in motif (6); and

(i) a substitution of valine as the ninth amino acid residue in motif (6).

When the modified phenylalanine dehydrogenase includes two or more mutations, one or more of the following combinations of mutations can be included:

(1) a combination of (f) the substitution of asparagine in motif (6) and (b) the substitution of threonine as the seventh amino acid residue in motif (2);

(c) the substitution of phenylalanine in motif (3);

(e) the substitution of arginine in motif (5);

(h) the substitution of glutamine in motif (6); or

(i) the substitution of valine as the ninth amino acid residue in motif (6);

(2) a combination of (c) the substitution of phenylalanine in motif (3) and (b) the substitution of threonine as the seventh amino acid residue in motif (2);

(g) the substitution of leucine in motif (6);

(h) the substitution of glutamine in motif (6); or

(i) the substitution of valine as the ninth amino acid residue in motif (6);

(3) a combination of (b) the substitution of threonine as the seventh amino acid residue in motif (2) and (g) the substitution of leucine in motif (6) or (h) the substitution of glutamine in motif (6); and

(4) a combination of (h) the substitution of glutamine in motif (6) and

(i) the substitution of valine as the ninth amino acid residue in motif (6).

When the modified phenylalanine dehydrogenase includes two or more mutations, one or more of the following mutations can be included:

(a) a substitution of leucine in motif (1) with tryptophane;

(b) a substitution of threonine as the seventh amino acid residue in motif (2) with serine;

(c) a substitution of phenylalanine in motif (3) with leucine or isoleucine;

(d) a substitution of arginine in motif (5) with aspartic acid;

(e) a substitution of asparagine in motif (6) with aspartic acid, methionine, glutamine, or cysteine;

(f) a substitution of leucine in motif (6) with glutamine or asparagine;

(g) a substitution of glutamine in motif (6) with aspartic acid; and

(h) a substitution of valine as the ninth amino acid residue in motif (6) with glycine, phenylalanine, or arginine.

When the modified phenylalanine dehydrogenase includes two or more mutations, one or more of the following combinations of mutations can be included:

(1) a combination of (f) the substitution of asparagine in motif (6) with aspartic acid, methionine, glutamine, or cysteine and (b) the substitution of threonine as the seventh amino acid residue in motif (2) with serine;

(c) the substitution of phenylalanine in motif (3) with leucine or isoleucine;

(e) the substitution of arginine in motif (5) with aspartic acid;

(h) the substitution of glutamine in motif (6) with aspartic acid; or

(i) the substitution of valine as the ninth amino acid residue in motif (6) with glycine, phenylalanine, or arginine;

(2) a combination of (c) the substitution of phenylalanine in motif (3) with leucine or isoleucine and (b) the substitution of threonine as the seventh amino acid residue in motif (2) with serine;

(g) the substitution of leucine in motif (6) with glutamine or asparagine;

(h) the substitution of glutamine in motif (6) with aspartic acid; or

(i) the substitution of valine as the ninth amino acid residue in motif (6) with glycine, phenylalanine, or arginine;

(3) a combination of (b) the substitution of threonine as the seventh amino acid residue in motif (2) with serine and (g) the substitution of leucine in motif (6) with glutamine or asparagine or (h) the substitution of glutamine in motif (6) with aspartic acid; and

(4) a combination of (h) the substitution of glutamine in motif (6) with aspartic acid and (i) the substitution of valine as the ninth amino acid residue in motif (6) with glycine, phenylalanine, or arginine.

When the modified phenylalanine dehydrogenase is one with three or more amino acid residues of the phenylalanine dehydrogenase mutated, at least one, at least two, and at least three of the mutations of the amino acid residues may be selected from the mutation of the amino acid residue described above. All the mutations of the amino acid residues may be selected from the mutation of the amino acid residue described above.

When the modified phenylalanine dehydrogenase includes three or more mutations, one or more of the following mutations can be included:

(a) a substitution of leucine in motif (1);

(b) a substitution of threonine as the seventh amino acid residue in motif (2);

(c) a substitution of phenylalanine in motif (3);

(e) a substitution of arginine in motif (5);

(f) a substitution of asparagine in motif (6); and

(i) a substitution of valine as the ninth amino acid residue in motif (6).

When the modified phenylalanine dehydrogenase includes three or more mutations, the following combination of mutations can be included:

a combination of (f) the substitution of asparagine in motif (6) and (b) the substitution of threonine as the seventh amino acid residue in motif (2), (c) the substitution of phenylalanine in motif (3), or (e) the substitution of arginine in motif (5).

When the modified phenylalanine dehydrogenase included three or more mutations, one or more of the following mutations can be included:

(a) a substitution of leucine in motif (1) with tryptophane;

(b) a substitution of threonine as the seventh amino acid residue in motif (2) with serine;

(c) a substitution of phenylalanine in motif (3) with isoleucine;

(e) a substitution of arginine in motif (5) with aspartic acid;

(f) a substitution of asparagine in motif (6) with aspartic acid or methionine; and

(i) a substitution of valine as the ninth amino acid residue in motif (6) with phenylalanine.

When the modified phenylalanine dehydrogenase includes three or more mutations, the following combination of mutations can be included:

a combination of (f) the substitution of asparagine in motif (6) with aspartic acid or methionine and (b) the substitution of threonine as the seventh amino acid residue in motif (2) with serine, (c) the substitution of phenylalanine in motif (3) with isoleucine, or

(e) the substitution of arginine in motif (5) with aspartic acid.

When the modified phenylalanine dehydrogenase is one with four or more amino acid residues of the phenylalanine dehydrogenase mutated, at least one, at least two, at least three, and at least four of the mutations of the amino acid residues may be selected from the mutation of the amino acid residue described above. All the mutations of the amino acid residues may be selected from the mutation of the amino acid residue described above.

When the modified phenylalanine dehydrogenase includes four or more mutations, one or more of the following mutations can be included:

(b) a substitution of threonine as the seventh amino acid residue in motif (2);

(c) a substitution of phenylalanine in motif (3);

(e) a substitution of arginine in motif (5); and

(f) a substitution of asparagine in motif (6).

When the modified phenylalanine dehydrogenase includes four or more mutations, the following combination of mutations can be included:

a combination of (f) the substitution of asparagine in motif (6) and

(b) the substitution of threonine as the seventh amino acid residue in motif (2),

(c) the substitution of phenylalanine in motif (3), or

(e) the substitution of arginine in motif (5).

When the modified phenylalanine dehydrogenase includes four or more mutations, one or more of the following mutations can be included:

(b) a substitution of threonine as the seventh amino acid residue in motif (2) with serine;

(c) a substitution of phenylalanine in motif (3) with isoleucine;

(e) a substitution of arginine in motif (5) with aspartic acid; and

(f) a substitution of asparagine in motif (6) with aspartic acid.

When the modified phenylalanine dehydrogenase includes four or more mutations, the following combination of mutations can be included:

a combination of (f) the substitution of asparagine in motif (6) with aspartic acid and

(b) the substitution of threonine as the seventh amino acid residue in motif (2) with serine,

(c) the substitution of phenylalanine in motif (3) with isoleucine, or

(e) the substitution of arginine in motif (5) with aspartic acid.

The phenylalanine dehydrogenase before the mutation may be a phenylalanine dehydrogenase having any of the following amino acid sequences (A) to (C).

(A) the amino acid sequence of SEQ ID NO:1,

(B) an amino acid sequence including substitution, deletion, insertion, or addition of one or several amino acid residues in the amino acid sequence of SEQ ID NO:1, or

(C) an amino acid sequence having 90% or more identity to the amino acid sequence of SEQ ID NO:1.

The amino acid sequence of SEQ ID NO:1 is a wild-type phenylalanine dehydrogenase derived from Thermoactinomyces intermedius (TiPheDH (wt)) and is encoded by a codon-optimized nucleotide sequence (SEQ ID NO:2) of TiPheDH (wt), for example.

The amino acid residue to be mutated, such as substitution, deletion, insertion, or addition, is normally L-alanine (A), L-asparagine (N), L-cysteine (C), L-glutamine (Q), L-isoleucine (I), L-leucine (L), L-methionine (M), L-phenylalanine (F), L-proline (P), L-serine (S), L-threonine (T), L-tryptophane (W), L-tyrosine (Y), L-valine (V), L-aspartic acid (D), L-glutamic acid (E), L-arginine (R), L-histidine (H), L-lysine (K), or glycine (G) as a natural L-α-amino acid. When the mutation is substitution, addition, or insertion, the amino acid residue to be substituted, added, or inserted is the same as the amino acid residue to be mutated described above. In the present specification, L and a may be omitted in the expression of the amino acid.

The amino acid sequence (B) described above may include mutations (for example, substitutions, deletions, insertions, and additions) of one or several amino acid residues. The number of the mutations is, for example, 1 to 50, 1 to 45, 1 to 40, 1 to 35, 1 to 30, or 1 to 25, 1 to 20, 1 to 15, and 1 to 10 (for example, 1, 2, 3, 4, 5, 6, 7, 8, 9, or 10).

The amino acid sequence (C) described above may have at least 90% or more amino acid sequence identity to the amino acid sequence of SEQ ID NO:1. The percentage of the amino acid sequence identity may be 91% or more, 92% or more, 93% or more, or 94% or more, 95% or more or 96% or more, 97% or more, and 98% or more or 99% or more.

A protein identified by the amino acid sequences (B) and (C) can have 50% or more, 60% or more, 70% or more, 80% or more, 90% or more, or 95% or more activity of the phenylalanine dehydrogenase activity of a protein having the amino acid sequence (A) described above when measured under the same conditions.

The amino acid sequence identity can be determined, for example, using algorithm BLAST by Karlin and Altschul (Pro. Natl. Acad. Sci. USA, 90, 5873 (1993)) and FASTA by Pearson (Methods Enzymol., 183, 63 (1990)). A program referred to as BLASTP has been developed based on this algorithm BLAST (see www.ncbi.nlm.nih.gov). Thus, the amino acid sequence identity may be calculated using these programs with default settings. Also, for example, a numerical value obtained by calculating Similarity as a percentage using a full-length polypeptide portion encoded in an ORF and using software GENETYX Ver 7.0.9 with a setting of “Gaps are NOT taken into account” or with a setting of Unit Size to Compare=2 from Genetyx Corporation employing Lipman-Pearson method may be used as the amino acid sequence identity. The lowest value among the values derived from these calculations may be employed as the amino acid sequence identity.

To prepare (B) the amino acid sequence including substitutions, deletions, insertions, or additions of one or several amino acid residues in the amino acid sequence of SEQ ID NO:1 and (C) the amino acid residue having 90% or more identity to the amino acid sequence of SEQ ID NO:1, when a mutation of an amino acid residue is introduced to the amino acid sequence of SEQ ID NO:1, and when the mutation of the amino acid residue is a substitution, such a substitution of the amino acid residue may be a conservative substitution. The term “conservative substitution” refers to substituting a certain amino acid residue with an amino acid residue having a similar side chain. Families of the amino acid residues having the similar side chain are well-known in the art. Examples of such families include amino acids having a basic side chain (for example, lysine, arginine, and histidine), amino acids having an acidic side chain (for example, aspartic acid and glutamic acid), amino acids having an uncharged polar side chain (for example, asparagine, glutamine, serine, threonine, tyrosine, and cysteine), amino acids having a nonpolar side chain (for example, glycine, alanine, valine, leucine, isoleucine, proline, phenylalanine, methionine, and tryptophane), amino acids having a β-position branched side chain (for example, threonine, valine, and isoleucine), amino acids having an aromatic side chain (for example, tyrosine, phenylalanine, tryptophane, and histidine), amino acids having a hydroxy group (for example, alcoholic, phenolic)-containing side chain (for example, serine, threonine, and tyrosine), and amino acids having a sulfur-containing side chain (for example, cysteine and methionine). The amino acid having an uncharged polar side chain and the amino acid having a nonpolar side chain may collectively be called a neutral amino acid. The conservative substitution of the amino acid may preferably be the substitution between aspartic acid and glutamic acid, the substitution among arginine, lysine, and histidine, the substitution between tryptophane and phenylalanine, the substitution between phenylalanine and valine, the substitution among leucine, isoleucine, and alanine, and the substitution between glycine and alanine.

When the phenylalanine dehydrogenase before the mutation is a phenylalanine dehydrogenase of (A) to (C) described above, the modified phenylalanine dehydrogenase may be a modified phenylalanine dehydrogenase including one or more of the following mutations:

R2, R10, Y11, C19, L41, G42, G43, C44, A50, S51, M66, C70, F77, K90, Y112, T115, D116, F124, R129, L137, K139, S140, K144, T147, K173, C200, C210, K216, K220, Q222, N227, R228, C234, C240, R255, C256, L257, N264, R271, Q277, K278, R279, S280, C282, N290, G293, L294, Q296, V297, R326, K328, N329, N331, C335, R340, K347, and/or K348, having enzyme activity, and showing improvement of one or more of the following characteristics: substrate specificity, solubility, and/or enzyme activity.

When the phenylalanine dehydrogenase before the mutation is a phenylalanine dehydrogenase of (A) to (C) described above, the modified phenylalanine dehydrogenase include one or more of the following substitutions:

R2D, R2E, R10D, R10E, Y11E, Y11D, C19A, C19S, L41W, L41F, L41Y, L41M, G42A, G43A, C44A, C44S, A50D, A50E, S51D, S51E, M66I, M66L, M66V, C70A, C70S, F77L, F77I, F77R, K90E, Y112L, T115S, D116E, F124L, F124I, R129K, L137V, K139E, S140A, K144G, T147A, T147S, T147N, K173E, K173D, C200A, C200S, C210S, C210A, K216D, K216E, K220D, K220E, Q222E, Q222D, N227D, N227E, R228E, R228D, C234A, C234S, C240S, C240A, R255E, R255D, C256A, C256S, L257K, N264G, N264Q, N264T, N264K, N264P, N264S, R271D, R271E, Q277D, K278D, K278E, R279D, R279E, S280D, C282S, C282A, N290V, N290D, N290M, N290Q, N290P, N290I, N290H, N290A, N290T, N290G, N290C, G293A, L294F, L294Q, L294H, L294N, L294I, L294D, L294G, L294E, L294T, L294S, Q296D, Q296E, Q296K, Q296N, Q296S, Q296R, V297Y, V297W, V297E, V297N, V297T, V297I, V297K, V297G, V297S, V297L, V297M, V297Q, V297F, V297C, V297R, R326E, K328E, K328D, N329D, N331E, N331D, C335A, C335S, R340D, R340E, K347D, and/or K348E, having enzyme activity, and showing improvement of one or more of the following characteristics substrate specificity, solubility, and/or enzyme activity.

In an embodiment, substrate specificity of the phenylalanine dehydrogenase for phenylalanine is improved. The improvement in the substrate specificity of the phenylalanine dehydrogenase for phenylalanine means that the reactivity of the modified phenylalanine dehydrogenase with phenylalanine further improves as compared with that of a wild-type enzyme. In other words, it means that the reactivity of the modified phenylalanine dehydrogenase with amino acids other than phenylalanine is reduced. Examples of the amino acids other than phenylalanine include L-α-amino acids other than phenylalanine. Specifically, examples of the L-α-amino acids other than phenylalanine include the 19 L-α-amino acids other than phenylalanine that form proteins, such as cystine, taurine, citrulline, ornithine, and α-amino butyric acid. Substrate specificity is measured by comparing the lowest reactivity of the phenylalanine dehydrogenase with an amino acid other than phenylalanine (for example, tyrosine) with the reactivity of phenylalanine dehydrogenase with phenylalanine (relative activity) as an indicator. The reactivity of the phenylalanine dehydrogenase may be measured based on the amount of NADH produced by an enzyme reaction. The extent of the improvement in the substrate specificity of the modified phenylalanine dehydrogenase with respect to the wild-type enzyme is more than 1 when the characteristic of the wild type is 1 and can be more than each of 1.01, 1.03, 1.04, 1.05, 1.1, 1.2, 1.3, 1.4, 1.5, 2.0, 3.0, 4.0, 5.0, 6.0, 7.0, 8.0, 9.0, 10.0, 11.0, and 12.0.

Preferred examples of a mutation suitable for the improvement in substrate specificity include the following:

(I) a mutation (for example, a substitution) of at least one amino acid residue in at least one of motifs (1) to (6);

(II) one or more of the following substitutions:

(a) a substitution of leucine in motif (1);

(b) a substitution of threonine as the seventh amino acid residue in motif (2);

(c) a substitution of phenylalanine in motif (3);

(d) a substitution of asparagine as the fourth amino acid residue in motif (4);

(e) a substitution of arginine in motif (5);

(f) a substitution of asparagine in motif (6);

(g) a substitution of leucine in motif (6);

(h) a substitution of glutamine in motif (6); and

(i) a substitution of valine as the ninth amino acid residue in motif (6); and

(III) one or more of the following mutations (for example, a substitution or substitutions):

R10, Y11, C19, L41, G42, G43, C44, M66, C70, F77, Y112, T115, D116, F124, R129, L137, K139, S140, K144, T147, K173, C200, C210, Q222, R228, C234, C240, R255, C256, N264, R271, K278, C282, N290, G293, L294, Q296, V297, and/or C335.

Further examples of the mutation suitable for the improvement in substrate specificity include the following:

(I) one or more of the following substitutions:

(a) a substitution of leucine in motif (1) with tryptophane, phenylalanine, tyrosine, or methionine;

(b) a substitution of threonine as the seventh amino acid residue in motif (2) with serine;

(c) a substitution of phenylalanine in motif (3) with leucine or isoleucine;

(d) a substitution of asparagine as the fourth amino acid residue in motif (4) with glycine, glutamine, threonine, lysine, proline, or serine;

(e) a substitution of arginine in motif (5) with aspartic acid;

(f) a substitution of asparagine in motif (6) with valine, aspartic acid, methionine, glutamine, proline, isoleucine, histidine, alanine, threonine, glycine, or cysteine;

(g) a substitution of leucine in motif (6) with phenylalanine, glutamine, histidine, asparagine, isoleucine, aspartic acid, glycine, glutamic acid, threonine, or serine;

(h) a substitution of glutamine in motif (6) with aspartic acid, glutamic acid, lysine, asparagine, serine, or arginine; and

(i) a substitution of valine as the ninth amino acid residue in motif (6) with tyrosine, tryptophane, glutamic acid, asparagine, threonine, isoleucine, lysine, glycine, serine, leucine, methionine, glutamine, phenylalanine, cysteine, or arginine, and

(II) one or more of the following mutations (for example, a substitution or substitutions):

R10D, Y11E, C19A, C19S, L41W, L41F, L41Y, L41M, G42A, G43A, C44A, C44S, M66I, M66L, M66V, C70A, C70S, F77L, F77I, F77R, Y112L, T115S, D116E, F124L, F124I, R129K, L137V, K139E, S140A, K144G, T147A, T147S, T147N, K173E, C200A, C210S, C210A, Q222D, R228E, C234A, C234S, C240S, C240A, R255E, C256A, C256S, N264G, N264Q, N264T, N264K, N264P, N264S, R271D, K278D, C282S, C282A, N290V, N290D, N290M, N290Q, N290P, N290I, N290H, N290A, N290T, N290G, N290C, G293A, L294F, L294Q, L294H, L294N, L294I, L294D, L294G, L294E, L294T, L294S, Q296D, Q296E, Q296K, Q296N, Q296S, Q296R, V297Y, V297W, V297E, V297N, V297T, V297I, V297K, V297G, V297S, V297L, V297M, V297Q, V297F, V297C, V297R, C335A, and/or C335S.

In another embodiment, solubility of the phenylalanine dehydrogenase is improved. The improvement in the solubility of the phenylalanine dehydrogenase means that the solubility of the modified phenylalanine dehydrogenase further improves as compared with that of a wild-type enzyme. Specifically, the solubility of the phenylalanine dehydrogenase can be measured with the concentration of supernatant fluid when an aqueous phenylalanine dehydrogenase solution is concentrated until it flocculates as an indicator, for example. The extent of the improvement in the solubility of the modified phenylalanine dehydrogenase with respect to the wild-type enzyme is more than 1 when the characteristic of the wild type is 1 and can be more than each of 1.01, 1.03, 1.04, 1.05, 1.1, 1.2, 1.3, 1.4, 1.5, 2.0, 3.0, 4.0, 5.0, 6.0, 7.0, 8.0, 9.0, 10.0, 11.0, and 12.0.

A mutation suitable for the improvement in solubility includes the following:

(I) a mutation (for example, a substitution) of at least one amino acid residue in at least one of motifs (1) to (3), (5), and (6);

(II) one or more of the following substitutions:

(a) a substitution of leucine in motif (1);

(b) a substitution of threonine as the seventh amino acid residue in motif (2);

(c) a substitution of phenylalanine in motif (3);

(e) a substitution of arginine in motif (5);

(f) a substitution of asparagine in motif (6); and

(i) a substitution of valine as the ninth amino acid residue in motif (6); and

(III) one or more of the following mutations (for example, a substitution or substitutions):

R2, R10, Y11, C19, L41, C44, A50, S51, C70, K90, T115, F124, K173, C200, C210, K216, K220, Q222, N227, R228, C240, R255, C256, L257, R271, Q277, K278, R279, C282, N290, V297, R326, N329, N331, C335, R340, and/or K348.

Further examples of the mutation suitable for the improvement in solubility include the following:

(I) one or more of the following substitutions:

(a) a substitution of leucine in motif (1) with tryptophane;

(b) a substitution of threonine as the seventh amino acid residue in motif (2) with serine;

(c) a substitution of phenylalanine in motif (3) with isoleucine;

(e) a substitution of arginine in motif (5) with aspartic acid or glutamic acid;

(f) a substitution of asparagine in motif (6) with aspartic acid or methionine; and

(i) a substitution of valine as the ninth amino acid residue in motif (6) with phenylalanine and

(II) one or more of the following mutations (for example, a substitution or substitutions):

R2D, R2E, R10D, R10E, Y11E, Y11D, C19A, C19S, L41W, C44A, C44S, A50D, A50E, S51D, S51E, C70A, C70S, K90E, T115S, F124I, K173E, K173D, C200A, C200S, C210S, C210A, K216D, K216E, K220D, K220E, Q222E, Q222D, N227D, N227E, R228E, R228D, C240S, C240A, R255E, R255D, C256A, L257K, R271D, R271E, Q277D, K278D, K278E, R279E, C282S, C282A, N290D, N290M, V297F, R326E, N329D, N331E, N331D, C335A, C335S, R340D, and/or K348E.

In still another embodiment, the activity of the phenylalanine dehydrogenase for phenylalanine is improved. The improvement in the activity of the phenylalanine dehydrogenase for phenylalanine means that the activity of the modified phenylalanine dehydrogenase for phenylalanine further improves as compared with that of a wild-type enzyme. Specifically, the improvement in the activity of the phenylalanine dehydrogenase for phenylalanine can be achieved when, with the activity of the wild-type phenylalanine dehydrogenase for phenylalanine at a certain concentration (for example, either concentration of a low concentration or a high concentration) being 100, the activity of the modified phenylalanine dehydrogenase for phenylalanine at the same concentration is larger than 100. Such a modified phenylalanine dehydrogenase enables quick and highly sensitive measurement of phenylalanine and is consequently useful for measurement of phenylalanine. The extent of the improvement in the activity of the modified phenylalanine dehydrogenase with respect to the wild-type enzyme is more than 1 when the characteristic of the wild type is 1 and can be more than each of 1.01, 1.03, 1.04, 1.05, 1.1, 1.2, 1.3, 1.4, 1.5, 2.0, 3.0, 4.0, 5.0, 6.0, 7.0, 8.0, 9.0, 10.0, 11.0, and 12.0.

Preferred examples of a mutation suitable for the improvement in activity include the following:

(I) a mutation (for example, a substitution) of at least one amino acid residue in at least one of motifs (3), (5), and (6);

(II) one or more of the following substitutions:

(c) a substitution of phenylalanine in motif (3);

(e) a substitution of arginine in motif (5);

(f) a substitution of asparagine in motif (6);

(g) a substitution of leucine in motif (6);

(h) a substitution of glutamine in motif (6); and

(i) a substitution of valine as the ninth amino acid residue in motif (6); and

(III) one or more of the following mutations (for example, a substitution or substitutions):

R10, Y11, C19, F77, F124, T147, K173, C200, C210, K216, K220, Q222, N227, R228, C234, C240, R255, C256, R271, K278, R279, S280, C282, N290, L294, Q296, V297, K328, N329, N331, C335, R340, and/or K347.

Further examples of the mutation suitable for the improvement in activity include the following:

(I) one or more of the following substitutions:

(c) a substitution of phenylalanine in motif (3) with isoleucine;

(e) a substitution of arginine in motif (5) with aspartic acid or glutamic acid;

(f) a substitution of asparagine in motif (6) with aspartic acid, threonine, or cysteine;

(g) a substitution of leucine in motif (6) with glutamine;

(h) a substitution of glutamine in motif (6) with aspartic acid or glutamic acid; and

(i) a substitution of valine as the ninth amino acid residue in motif (6) with threonine or glycine and

(II) one or more of the following mutations (for example, a substitution or substitutions):

R10D, Y11E, C19S, F77L, F124I, T147S, K173E, K173D, C200A, C200S, C210S, K216D, K216E, K220D, K220E, Q222E, Q222D, N227D, N227E, R228E, R228D, C234A, C234S, C240S, C240A, R255E, R255D, C256A, R271D, R271E, K278D, R279D, R279E, S280D, C282S, N290D, N290T, N290C, L294Q, Q296D, Q296E, V297T, V297G, K328E, K328D, N329D, N331D, C335A, R340E, and/or K347D.

The modified phenylalanine dehydrogenase may be one having the mutation described above alone or both the mutation described above and a supplemental mutation to include an amino acid sequence having at least 90% or more amino acid sequence identity to the amino acid sequence of the (wild-type) phenylalanine dehydrogenase before the mutation. The percentage of the amino acid sequence identity may be 92% or more, 95% or more, 97% or more, and 98% or more or 99% or more.

The modified phenylalanine dehydrogenase may also have another peptide component (for example, a tag moiety) at a C-terminus or an N-terminus. Examples of the other peptide component that can be added to the modified phenylalanine dehydrogenase can include peptide components making purification of an objective protein easy (for example, tag moiety such as histidine tag and strep-tag II; proteins such as glutathione-S-transferase and maltose-binding protein commonly used for the purification of the objective protein), peptide components improving the solubility of the objective protein (for example, Nus-tag), peptide components working as a chaperon (for example, trigger factor), and peptide components as a protein or a domain of the protein having another function or a linker connecting them.

The amino acid sequence identity can be determined, for example, using algorithm BLAST by Karlin and Altschul (Pro. Natl. Acad. Sci. USA, 90, 5873 (1993)) and FASTA by Pearson (Methods Enzymol., 183, 63 (1990)). A program referred to as BLASTP has been developed based on this algorithm BLAST (see www.ncbi.nlm.nih.gov). Thus, the amino acid sequence identity may be calculated using these programs with default settings. Also, for example, a numerical value obtained by calculating Similarity as a percentage using a full length polypeptide portion encoded in an ORF and using software GENETYX Ver 7.0.9 with a setting of Unit Size to Compare=2 from Genetyx Corporation employing Lipman-Pearson method may be used as the amino acid sequence identity. The lowest value among the values derived from these calculations may be employed as the amino acid sequence identity.

The position of an amino acid residue at which the supplemental mutation can be introduced in an amino acid sequence is apparent to a person skilled in the art; the supplemental mutation can be introduced with reference to alignment of amino acid sequences, for example. Specifically, a person skilled in the art can 1) compare amino acid sequences of a plurality of homologs (for example, the amino acid sequence of SEQ ID NO:1 and an amino acid sequence of the other homolog) with each other, (2) demonstrate relatively conserved regions and relatively not conserved regions, then (3) predict regions capable of playing a functionally important role and regions incapable of playing a functionally important role from the relatively conserved regions and the relatively not conserved regions, respectively, and thus recognize correlativity between a structure and a function. The analysis result of the three-dimensional structure has been reported for phenylalanine dehydrogenases as described above, and thus a person skilled in the art can introduce the supplemental mutation based on the analysis result of the three-dimensional structure so as to enable the retention of the characteristics described above. The site at which the supplemental mutation is introduced may be an amino acid residue other than the amino acid residue described above.

When the supplemental mutation of the amino acid residue is a substitution, such a substitution of the amino acid residue may be a conservative substitution. The term “conservative substitution” refers to substituting a certain amino acid residue with an amino acid residue having a similar side chain. Families of the amino acid residues having the similar side chain are well-known in the art. Examples of such families comprise amino acids having a basic side chain (for example, lysine, arginine, and histidine), amino acids having an acidic side chain (for example, aspartic acid and glutamic acid), amino acids having an uncharged polar side chain (for example, asparagine, glutamine, serine, threonine, tyrosine, and cysteine), amino acids having a nonpolar side chain (for example, glycine, alanine, valine, leucine, isoleucine, proline, phenylalanine, methionine, and tryptophane), amino acids having a β-position branched side chain (for example, threonine, valine, and isoleucine), amino acids having an aromatic side chain (for example, tyrosine, phenylalanine, tryptophane, and histidine), amino acids having a hydroxy group (for example, alcoholic, phenolic)-containing side chain (for example, serine, threonine, and tyrosine), and amino acids having a sulfur-containing side chain (for example, cysteine and methionine). The conservative substitution of the amino acid may preferably be the substitution between aspartic acid and glutamic acid, the substitution among arginine, lysine, and histidine, the substitution between tryptophane and phenylalanine, the substitution between phenylalanine and valine, the substitution among leucine, isoleucine, and alanine, and the substitution between glycine and alanine.

The present invention also provides a polynucleotide encoding the modified phenylalanine dehydrogenase. The polynucleotide may be DNA or RNA.

The modified phenylalanine dehydrogenase can be prepared using a transformant expressing the modified phenylalanine dehydrogenase or a cell-free system. The transformant can be produced by producing an expression vector and then introducing this expression vector into a host, for example.

The expression vector can include the polynucleotide encoding the modified phenylalanine dehydrogenase.

The expression vector comprises the polynucleotide (for example, DNA or RNA) encoding the modified phenylalanine dehydrogenase. The expression vector can further include regions encoding a promoter, a terminator, and a drug (for example, tetracycline, ampicillin, kanamycin, hygromycin, or phosphinothricin) resistant gene in addition to the polynucleotide as described herein. The expression vector may be a plasmid or an integrative vector. The expression vector may be a virus vector or a vector for a cell-free system. The expression vector may further include a polynucleotide encoding another peptide component that can be added to the modified phenylalanine dehydrogenase at the 3′ or 5′-end side with respect to the polynucleotide. Examples of the polynucleotide encoding the other peptide component include a polynucleotide encoding the peptide component that renders purification of an objective protein easy as described above, a polynucleotide encoding the peptide component improving the solubility of the objective protein as described above, a polynucleotide encoding the peptide component working as a chaperon, and a polynucleotide encoding the peptide component as a protein or a domain of the protein having another function or a linker connecting them. Various expression vectors including the polynucleotide encoding the other peptide component can be used. Consequently, to produce the expression vector, such expression vectors may be used. Examples thereof include expression vectors including the polynucleotide encoding the peptide component rendering purification of an objective protein easy (for example, pET-15b, pET-51b, pET-41a, and pMAL-p5G), expression vectors including the polynucleotide encoding the peptide component improving the solubility of the objective protein (for example, pET-50b), expression vectors including the polynucleotide encoding the peptide component working as a chaperon (for example, pCold TF), and expression vectors including the polynucleotide encoding the peptide component as a protein or a domain of the protein having another function or a linker connecting them. To enable cleavage between the modified phenylalanine dehydrogenase and the other peptide component added thereto after protein expression, the expression vector may include a region encoding a site to be cleaved by a protease between the polynucleotide encoding the modified phenylalanine dehydrogenase and the polynucleotide encoding the other peptide component.

Various prokaryotic cells including bacteria belonging to the genus Escherichia such as Escherichia coli, bacteria belonging to the genus Corynebacterium (for example, Corynebacterium glutamicum), and bacteria belonging to the genus Bacillus (for example, Bacillus subtilis) and various eukaryotic cells including fungi belonging to the genus Saccharomyces (for example, Saccharomyces cerevisiae), fungi belonging to the genus Pichia (for example, Pichia stipitis), and fungi belonging to the genus Aspergillus (for example, Aspergillus oryzae) can be used as the host for expressing the modified phenylalanine dehydrogenase. A strain in which a certain gene has been deleted may be used as the host. Examples of the transformant may include transformants in which the expression vector is retained in its cytoplasm and transformants in which an objective gene is integrated into its genome.

The transformant is a host cell that can produce the modified phenylalanine dehydrogenase or can express the polynucleotide to produce the modified phenylalanine dehydrogenase. Specifically, the transformant is a host cell including an expression unit including the polynucleotide. Examples of the host cell including the expression unit including the polynucleotide include a host cell into which the expression vector is introduced as a whole and a host cell in which an expression unit in the expression vector is introduced to its genome. The host cell is not limited to a particular host cell so long as it can express the modified phenylalanine dehydrogenase. The host cell may be homologous or heterologous with respect to the modified phenylalanine dehydrogenase and the polynucleotide and can be heterologous therewith. The host cell may be homologous or heterologous with respect to the promoter and can be heterologous therewith. Examples of the host cell include animal cells, plant cells, insect cells, and microorganisms; microorganisms are a particular example. The host cell for use can be a bacterium or a fungus. The bacterium may be a Gram-positive bacterium or a Gram-negative bacterium.

The transformant can be cultured in a medium having a composition described below, for example, using a certain culture apparatus (for example, a test tube, a flask, or a jar fermenter). The culture conditions can be set as appropriate. Specifically, the culture temperature may be 10° C. to 37° C., pH may be 6.5 to 7.5, and the culture time may be 1 hour to 100 hours. Culture may be performed while controlling a dissolved oxygen concentration. In this case, the dissolved oxygen concentration (a DO value) in a culture liquid may be used as an indicator for the control. Ventilation and stirring conditions can be controlled such that a relative dissolved oxygen concentration DO value when the oxygen concentration in the atmosphere is 21% will not be less than 1 to 10%, for example, 3% to 8%. Culture may be batch culture or fed-batch culture. In the case of the fed-batch culture, culture can be continued by adding a solution as a sugar source and a solution containing phosphoric acid to the culture liquid continuously or continually in a successive manner.

The host to be transformed is as described above; describing Escherichia coli in detail, the host can be selected from Escherichia coli K12 subspecies Escherichia coli JM109 strain, DH5a strain, HB101 strain, BL21 (DE3) strain, and the like. The method for performing transformation and the method for selecting the transformant are described in Molecular Cloning: A Laboratory Manual, 3rd edition, Cold Spring Harbor press (2001 Jan. 15) and the like. The following describes a method for producing a transformed Escherichia coli and producing a certain enzyme using the same more specifically as an example.

A promoter generally used for producing a foreign protein in E. coli can be used as a promoter for expressing the polynucleotide; examples thereof include potent promoters such as PhoA, PhoC, a T7 promoter, a lac promoter, a trp promoter, a trc promoter, a tac promoter, a PR promoter and a PL promoter of the lambda phage, and a T5 promoter, and PhoA, PhoC, and lac are particular examples. For example, pUC (for example, pUC19 and pUC18), pSTV, pBR (for example, pBR322), pHSG (for example, pHSG299, pHSG298, pHSG399, and pHSG398), RSF (for example, RSF1010), pACYC (for example, pACYC177 and pACYC184), pMW (for example, pMW119, pMW118, pMW219, and pMW218), pQE (for example, pQE30), and derivatives thereof may be used as a vector. A vector from phage DNA may also be utilized as the other vector. Furthermore, an expression vector that includes a promoter and can express an inserted DNA sequence may also be used. The vector may be pUC, pSTV, or pMW.

Also, a terminator that is a transcription terminating sequence may be ligated downstream of the polynucleotide. Examples of such a terminator include a T7 terminator, an fd phage terminator, a T4 terminator, a terminator of a tetracycline resistant gene, and a terminator of Escherichia coli trpA gene.

The vector for introducing the polynucleotide into Escherichia coli can be a so-called multicopy type; examples thereof include plasmids having a replication starting point derived from ColE1 such as pUC-based plasmids, pBR322-based plasmids, and derivatives thereof. The “derivative” can one in which modification has been performed on the plasmid by substitution, deletion, insertion, and/or addition of nucleotides.

To select the transformant, the vector can have a marker such as an ampicillin resistant gene. Expression vectors having a potent promoter are commercially available as such a plasmid (for example, pUC-based ones (manufactured by Takara Bio Inc.), pPROK-based ones (manufactured by Clontech), and pKK233-2 (manufactured by Clontech)).

The modified phenylalanine dehydrogenase can be obtained by transforming Escherichia coli using the obtained expression vector and culturing this Escherichia coli.

Media such as M9-casamino acid medium and LB medium generally used for culturing Escherichia coli may be used as the medium. The medium may contain a certain carbon source, nitrogen source, and coenzyme (for example, pyridoxine hydrochloride). Specifically, peptone, yeast extract, NaCl, glucose, MgSO₄, ammonium sulfate, potassium dihydrogen phosphate, ferric sulfate, manganese sulfate, and the like may be used. Culture conditions and production guidance conditions can be selected as appropriate in accordance with the types of a marker and a promoter of the chosen vector, a host bacterium, and the like.

The modified phenylalanine dehydrogenase is collected by the following methods or the like. The modified phenylalanine dehydrogenase can be obtained as a pulverized product or a dissolved product by collecting the transformant and then pulverizing (for example, sonication or homogenization) or dissolving (for example, lysozyme treatment) bacterial cells. The modified phenylalanine dehydrogenase can be obtained by subjecting such a pulverized product or dissolved product to a method such as extraction, precipitation, filtration, or column chromatography.

A method for analyzing phenylalanine is provided. The method of analysis can include measuring phenylalanine contained in a test sample using the modified phenylalanine dehydrogenase.

The test sample is not limited to a particular test sample so long as it is a sample suspected to contain phenylalanine; examples thereof include samples derived from living bodies (for example, blood, urine, saliva, and tear) and food and drink (for example, nutritional drinks and amino acid drinks). Phenylalanine in the test sample may be in a low concentration (for example, a concentration of less than 1 mM such as 1 μM or more and less than 1 mM) or in a high concentration (for example, a concentration of 1 mM or more such as 1 mM or more and less than 1 M).

The method of analysis is not limited to a particular method so long as it can measure phenylalanine using the modified phenylalanine dehydrogenase; phenylalanine is measured by mixing the test sample with nicotinamide adenine dinucleotide(NAD⁺) in an alkaline or neutral condition, or preferably in an alkaline buffer solution, then subjecting the mixed sample to an enzyme reaction using the modified phenylalanine dehydrogenase, and lastly detecting NADH produced from NAD⁺ by the action of the modified phenylalanine dehydrogenase, for example. Specifically, in the presence of nicotinamide adenine dinucleotide(NAD⁺), in an alkaline buffer solution, the modified phenylalanine dehydrogenase acts on the test sample, whereby an amino group of a substrate contained in a sample derived from a living body is subjected to oxidative deamination, and the reduced form (NADH) is produced from nicotinamide adenine dinucleotide(NAD⁺). Thus, quantification of phenylalanine can be performed by detecting NADH through absorbance (340 nm) or the like. The method for measuring amino acids based on such methodology are known (for example, refer to Ueatrongchit T, Asano Y, Anal Biochem., 2011 Mar. 1; 410(1): 44-56). Quantification of phenylalanine can also be performed by reducing a dye with the produced NADH and detecting the color development of the reduced dye as absorbance or the like. Furthermore, NADH can also be detected by an electrochemical method. Phenylalanine can be measured by electrochemically oxidizing NADH produced by causing the modified phenylalanine dehydrogenase to act on the test sample in an alkaline or neutral condition and measuring its oxidation current, or alternatively, by reducing a coexisting electron mediator by the produced NADH and measuring an electrochemical oxidation current of the reduced electron mediator, for example. Electron transfer between NADH and the electron mediator may be mediated by a catalyst. Note that measurement of phenylalanine can be performed by the rate method (the initial rate method).

The modified phenylalanine dehydrogenase does not react with amino acids other than phenylalanine or has low reactivity therewith. Consequently, even when not only phenylalanine but also other amino acids are contained in the test sample, the amount of phenylalanine in the test sample can be evaluated using the modified phenylalanine dehydrogenase.

Furthermore, a kit for analyzing phenylalanine including the modified phenylalanine dehydrogenase is described.

The kit can further include at least one of a buffer solution or a buffer salt for reaction and nicotinamide adenine dinucleotide (NAD⁺).

The buffer solution or the buffer salt for reaction is used for maintaining pH of a reaction liquid at a value suitable for an objective enzyme reaction. The buffer solution or the buffer salt for reaction is alkaline or neutral, for example, and preferably alkaline.

When the kit includes nicotinamide adenine dinucleotide(NAD⁺), the kit may further include a dye to be reduced by NADH. In this case, the dye is reduced by NADH produced from NAD⁺ by the action of the modified phenylalanine dehydrogenase, and the color development of the reduced dye can be detected by absorbance or the like. A substance working as an electron mediator may be involved in the reduction of the dye.

An enzyme sensor for analyzing phenylalanine including (a) an electrode for detection and (b) the modified phenylalanine dehydrogenase as described herein immobilized or disposed on the electrode for detection. The modified phenylalanine dehydrogenase is immobilized or disposed on the electrode directly or indirectly.

As the electrode for detection, it is possible to use a biosensor directly or indirectly detecting a product or a byproduct (NH₃+NADH+H⁺) produced from phenylalanine by the modified phenylalanine dehydrogenase, for example; more specific examples include an electrode for detection utilizing the modified phenylalanine dehydrogenase and nicotinamide adenine dinucleotide(NAD⁺). As such an electrode for detection, those described in WO 2005/075970 and WO 00/57166 can be used, for example.

### EXAMPLES

The following describes the present invention with reference to the following examples in more detail; the present invention is not limited to the following examples.

### Example 1 Construction of Plasmid for Expressing PheDH (Wild Type)

A recombinant expression system of PheDH using Escherichia coli was constructed. First, a plasmid for recombinant expression was constructed. As a sequence for insertion to pET-24a (Merck), in a nucleotide sequence (codon-optimized PheDH, SEQ ID NO:2) encoding the amino acid sequence of wild-type PheDH derived from Thermoactinomyces intermedius (SEQ ID NO:1), a DNA fragment (SEQ ID NO:112) in which a nucleotide sequence including an NdeI site+an initiation codon+a His-tag code sequence (CATATGCATCACCATCACCACCAC, SEQ ID NO:113) is added to the 5′-end, whereas a nucleotide sequence including a termination codon+a BamHI site (TAATGAGGATCC, SEQ ID NO:114) is added to the 3′-end was produced by chemical synthesis, which was incorporated into restriction enzyme sites of NdeI and BamHI of pET-24a to obtain a plasmid for expressing PheDH (the wild type). Using a standard method for analyzing a DNA sequence with this plasmid as a template, insertion of an objective gene to the plasmid was confirmed. In accordance with a standard method, a transformant of Escherichia coli BL21 (DE3) was acquired.

In the following, the plasmid including the PheDH sequence with the His-tag added to the N-terminus (SEQ ID NO:111 for the amino acid sequence and SEQ ID NO:112 for the nucleotide sequence) (the plasmid for expressing PheDH) will be called pET24a-PheDH, whereas the transformant of BL21 (DE3) by pET24a-PheDH will be called pET24a-PheDH-BL21 (DE3).

### Example 2 Construction of Plasmid for Expressing PheDH Mutant

A PheDH mutant was prepared as follows. Using KAPA HiFi HS ReadyMix (Kapa Biosystems, Inc.), in accordance with a standard method with pET24a-PheDH as a template, mutation introduction to a PheDH gene was performed. When a plurality of mutations were introduced, supplemental mutations were successively introduced using a plasmid with a mutation introduced as a template. Using a standard method for analyzing a DNA sequence with each expression plasmid as a template, introduction of an objective mutation to the plasmid was confirmed. In accordance with a standard method, a transformant of Escherichia coli BL21 (DE3) was acquired.

### Example 3 Preparation of PheDH

### Preparation of PheDH for Evaluating Substrate Specificity

Preparation of PheDH for evaluating substrate specificity was performed as follows. First, from a glycerol stock of the transformant of Escherichia coli BL21 (DE3) acquired in Example 1 and Example 2, the bacteria were inoculated into an LB plate containing 25 μg/mL of kanamycin and were stationarily cultured at 37° C. overnight. An LB liquid medium containing 25 μg/mL of kanamycin in an amount of 2 mL was put into a tube with a volume of 14 mL, and a single colony on the LB plate was inoculated and was cultured by reciprocal shaking at 37° C. overnight. A culture liquid in an amount of 50 μL was added to 4 mL of the LB liquid medium containing 25 μg/mL of kanamycin and was cultured by reciprocal shaking until the value of OD600 at 37° C. reached about 0.9. The culture liquid was left at rest at 30° C. for 30 minutes, isopropyl β-D-thiogalactopyranoside (IPTG) was added thereto so as to have a final concentration of 0.5 mM, the culture liquid was cultured by reciprocal shaking at 30° C. overnight, and then the bacteria were collected into a 2 mL tube.

Bacterium cells were suspended in a buffer for pulverization (200 mM Tris-HCl, pH 8.0) and were pulverized using an ultrasonic pulverizer (BIORUPTOR manufactured by Cosmo Bio Co., Ltd.). This pulverized liquid was centrifuged at 6,000×g for 10 minutes to collect PheDH as an objective protein as a supernatant.

### Preparation of PheDH for Evaluating Solubility and Activity

Preparation of PheDH for evaluating solubility and activity was performed as follows. First, from a glycerol stock of the transformant of Escherichia coli BL21 (DE3) acquired in Example 1 and Example 2, the bacteria were inoculated into an LB plate containing 25 μg/mL of kanamycin and were stationarily cultured at 37° C. overnight. An LB liquid medium containing 25 μg/mL of kanamycin in an amount of 2 mL was put into a tube with a volume of 14 mL, and a single colony on the LB plate was inoculated and was cultured by reciprocal shaking at 37° C. overnight. A culture liquid in an amount of 300 μL was added to 30 mL of the LB liquid medium containing 25 μg/mL of kanamycin and was cultured by reciprocal shaking until the value of OD600 at 37° C. reached about 0.9. The culture liquid was left at rest at 30° C. for 30 minutes, IPTG was added thereto so as to have a final concentration of 0.5 mM, the culture liquid was cultured by reciprocal shaking at 30° C. overnight, and then the bacteria were collected into a 50 mL tube.

Bacterium cells were suspended in a buffer for wash (50 mM HEPES, 500 mM NaCl, 50 mM imidazole, pH 7.5) and were pulverized using an ultrasonic pulverizer (BIORUPTOR manufactured by Cosmo Bio Co., Ltd.). This pulverized liquid was centrifuged at 14,000×g for 10 minutes, and the supernatant was collected, which was then added to Ni Sepharose 6 Fast Flow (manufactured by GE Healthcare Japan Corporation) equilibrated with the buffer for wash and was subjected to mild mixing with inversion at room temperature for 5 minutes, and the solution was removed by free fall using EconoSpin (trademark) Empty Column (manufactured by GeneDesign, Inc.). Subsequently, after being washed with the buffer for wash, PheDH as an objective protein was eluted with an elution buffer (50 mM HEPES, 500 mM NaCl, 500 mM imidazole, pH 7.5). The solvent of the PheDH solution was replaced with a buffer for stock (100 mM Tris-HCl, pH 8.0) by ultrafiltration.

### Example 4 Evaluation of Substrate Specificity of PheDH

Evaluation of the substrate specificity of each of the enzymes prepared in Example 3 was performed in accordance with the following procedure. PheDH was prepared so as to be 0.5 mg/mL, and a change with the lapse of time of the absorbance at a wavelength of 340 nm of a solution obtained by adding 100 μL of 200 mM Glycine-KCl-KOH with PH 10.0, 4 μL of 50 mM NAD⁺ (manufactured by Fujifilm Wako Pure Chemical Corporation), 20 μL of a 10 mM aqueous L-phenylalanine solution or a 10 mM aqueous L-tyrosine solution, and 56 μL of ultrapure water to 20 μL of PheDH was measured with a microplate reader (SpectraMax M2e manufactured by Molecular Devices) for 5 minutes. Tables 8 and 9 list relative activity at the time of measurement of the aqueous L-tyrosine solution with an absorbance value 5 minutes after the time of measurement of the aqueous L-phenylalanine solution being 100% for each of the wild type and mutants. The results of Tables 8 and 9 were calculated from an average value when an experience was conducted twice for the same sample. For the value of WT, an average value of results of the wild-type PheDH prepared and subjected to substrate specificity evaluation 16 times was used. When a mutated PheDH with multiple mutations introduced is indicated, the introduced mutations are sectioned by/to be continuously described. C19S/N290D, for example, means that it is a mutated PheDH having two mutations of C19S and N290D. WT means that it is of the wild type.

It can be seen from the result of Table 8 that introduction of the mutations listed in Table 8 can inhibit the reactivity of PheDH with L-tyrosine. Furthermore, it can be seen from the result of Table 9 that introduction of the multiple mutations listed in Table 9 can inhibit the reactivity of PheDH with L-tyrosine.

TABLE 8
Substrate specificity of wild-type and single mutation-introduced PheDH

| Relative activity for Tyr | | Relative activity for Tyr | | Relative activity for Tyr | |
|---|---|---|---|---|---|
| with Phe being 100% | | with Phe being 100% | | with Phe being 100% | |
| WT | 56.0% | C240S | 47.4% | L294G | 35.0% |
| L41W | 7.1% | C240A | 48.3% | L294E | 41.2% |
| L41F | 11.0% | N264G | 13.3% | L294T | 41.7% |
| L41Y | 12.9% | N264Q | 20.0% | L294S | 45.3% |
| L41M | 46.1% | N264T | 23.5% | Q296D | 22.5% |
| G42A | 27.3% | N264K | 26.8% | Q296E | 29.3% |
| G43A | 24.3% | N264P | 27.5% | Q296K | 36.1% |
| M66I | 19.4% | N264S | 39.3% | Q296N | 36.1% |
| M66L | 19.7% | N290V | 8.5% | Q296S | 37.6% |
| M66V | 24.2% | N290D | 10.5% | Q296R | 41.4% |
| F77L | 29.8% | N290M | 11.4% | V297Y | 11.1% |
| F77I | 37.7% | N290Q | 11.4% | V297W | 15.7% |
| F77R | 41.4% | N290P | 11.7% | V297E | 15.8% |
| Y112L | 47.8% | N290I | 13.1% | V297N | 17.7% |
| T115S | 24.0% | N290H | 14.4% | V297T | 18.5% |
| D116E | 39.9% | N290A | 20.8% | V297I | 21.4% |
| F124L | 20.2% | N290T | 29.2% | V297K | 27.5% |
| F124I | 26.9% | N290G | 43.2% | V297G | 27.8% |
| R129K | 48.3% | N290C | 49.0% | V297S | 29.9% |
| L137V | 37.0% | G293A | 28.0% | V297L | 31.6% |
| K139E | 44.3% | L294F | 10.2% | V297M | 34.8% |
| S140A | 49.8% | L294Q | 15.5% | V297Q | 37.3% |
| K144G | 39.1% | L294H | 19.3% | V297F | 40.6% |
| T147A | 20.5% | L294N | 22.4% | V297C | 41.5% |
| T147S | 28.9% | L294I | 23.2% | V297R | 47.1% |
| T147N | 35.2% | L294D | 24.7% | | |

TABLE 9
Substrate specificity of wild-type and multiple mutations-introduced PheDH

| Relative activity for Tyr with Phe | | Relative activity for Tyr with Phe | |
|---|---|---|---|
| being 100% | | being 100% | |
| WT | 56.0% | Y11E/R271D/N290D | 13.4% |
| R10D/N290D | 7.1% | Y11E/K278D/N290M | 4.9% |
| Y11E/N290D | 6.5% | Y11E/K278D/N290D | 5.7% |
| C19S/N290D | 7.7% | Y11E/K278D/V297F | 9.0% |
| C19A/N290D | 11.0% | L41W/K173E/K278D | 9.0% |
| C44S/N290D | 11.2% | L41W/R228E/K278D | 5.8% |
| C44A/N290D | 14.5% | K173E/K278D/N290M | 4.4% |
| C70A/N290D | 9.9% | K173E/K278D/N290D | 4.7% |
| C70S/N290D | 13.0% | K173E/K278D/V297F | 6.9% |
| T115S/F124L | 24.0% | R228E/K278D/N290M | 6.6% |
| T115S/N290C | 7.7% | R228E/K278D/N290D | 4.9% |
| T115S/N290Q | 13.5% | R228E/K278D/V297F | 5.8% |
| T115S/L294Q | 11.0% | R10D/Y11E/K173E/N290D | 5.9% |
| T115S/L294N | 12.0% | R10D/Y11E/Q222D/N290D | 7.0% |
| T115S/Q296D | 12.9% | R10D/Y11E/R228E/N290D | 5.9% |
| F124L/N290C | 10.7% | R10D/Y11E/R255E/N290D | 6.4% |
| F124L/N290Q | 12.0% | R10D/Y11E/K278D/N290D | 6.3% |
| F124I/N290D | 27.6% | R10D/T115S/Q222D/N290D | 1.9% |
| F124L/L294N | 20.5% | R10D/T115S/R255E/N290D | 1.2% |
| F124L/L294Q | 25.2% | R10D/F124I/Q222D/N290D | 3.2% |
| F124L/Q296D | 11.2% | R10D/F124I/R255E/N290D | 2.5% |
| F124L/V297R | 33.6% | R10D/K173E/Q222D/N290D | 5.3% |
| T147S/N290D | 18.0% | R10D/K173E/R228E/N290D | 4.6% |
| K173E/N290D | 6.3% | R10D/K173E/R255E/N290D | 5.4% |
| C200A/N290D | 24.6% | R10D/K173E/R271D/N290D | 5.8% |
| C210A/N290D | 15.5% | R10D/K173E/K278D/N290D | 5.1% |
| C210S/N290D | 20.6% | R10D/Q222D/R228E/N290D | 5.2% |
| Q222D/N290D | 6.0% | R10D/Q222D/R255E/N290D | 5.5% |
| R228E/N290D | 4.6% | R10D/Q222D/R271D/N290D | 4.4% |
| C234A/N290D | 24.0% | R10D/Q222D/K278D/N290D | 6.1% |
| C234S/N290D | 35.8% | R10D/R228E/R255E/N290D | 5.4% |
| C240S/N290D | 41.0% | R10D/R228E/K278D/N290D | 5.3% |
| C240A/N290D | 49.8% | R10D/R255E/R271D/N290D | 4.9% |
| R255E/N290D | 5.5% | R10D/R255E/K278D/N290D | 7.2% |
| C256A/N290D | 18.4% | Y11E/T115S/R228E/N290D | 2.5% |
| C256S/N290D | 25.2% | Y11E/T115S/K278D/N290D | 4.8% |
| R271D/N290D | 10.8% | Y11E/F124I/R228E/N290D | 3.0% |
| K278D/N290D | 6.5% | Y11E/F124I/K278D/N290D | 3.1% |
| C282A/N290D | 5.3% | Y11E/K173E/Q222D/N290D | 6.2% |
| C282S/N290D | 12.6% | Y11E/K173E/R228E/N290D | 6.1% |
| N290C/Q296D | 13.7% | Y11E/K173E/R255E/N290D | 6.1% |
| N290D/V297G | 16.6% | Y11E/K173E/R271D/N290D | 5.0% |
| N290D/C335A | 8.6% | Y11E/K173E/K278D/N290D | 5.5% |
| N290D/C335S | 8.7% | Y11E/Q222D/R228E/N290D | 6.1% |
| Q296D/V297R | 36.6% | Y11E/Q222D/K278D/N290D | 6.4% |
| R10D/L41W/Q222D | 5.5% | Y11E/R228E/R255E/N290D | 7.2% |
| R10D/L41W/R255E | 7.2% | Y11E/R228E/R271D/N290D | 6.1% |
| R10D/K173E/N290D | 3.8% | Y11E/R228E/K278D/N290D | 6.1% |
| R10D/Q222D/N290M | 4.6% | Y11E/R255E/K278D/N290D | 6.1% |
| R10D/Q222D/N290D | 4.6% | Y11E/R271D/K278D/N290D | 5.5% |
| R10D/Q222D/V297F | 7.1% | T115S/K173E/K278D/N290D | 4.4% |
| R10D/R255E/N290M | 4.8% | T115S/R228E/K278D/N290D | 1.5% |
| R10D/R255E/N290D | 4.8% | F124I/K173E/K278D/N290D | 4.1% |
| R10D/R255E/V297F | 7.6% | F124I/R228E/K278D/N290D | 2.8% |
| R10D/R271D/N290D | 11.4% | K173E/Q222D/K278D/N290D | 4.2% |
| Y11E/L41W/R228E | 6.5% | K173E/R228E/K278D/N290D | 4.2% |
| Y11E/L41W/K278D | 5.6% | K173E/R255E/K278D/N290D | 5.1% |
| Y11E/K173E/N290D | 5.2% | K173E/R271D/K278D/N290D | 4.5% |
| Y11E/R228E/N290M | 5.7% | Q222D/R228E/K278D/N290D | 4.7% |
| Y11E/R228E/N290D | 5.0% | R228E/R255E/K278D/N290D | 5.5% |
| Y11E/R228E/V297F | 7.2% | R228E/R271D/K278D/N290D | 5.5% |

### Example 5 Evaluation of Solubility of PheDH

Evaluation of the solubility of each of the enzymes prepared in Example 3 was performed as follows. Concentration was performed by ultrafiltration until PheDH the solvent of which had been replaced with a buffer for stock flocculated, and 30 μL of PheDH was put into a tube with a volume of 1.5 mL and was centrifuged at 18,500×g for 60 minutes, and the supernatant was collected. The concentration of the collected supernatant after centrifugation of the wild type and mutants was determined to be solubility. Table 10 and Table 11 list results compared with the solubility of the wild-type PheDH.

It can be seen from the result of Table 10 that introduction of the mutations listed in Table 10 increases the solubility of PheDH. Furthermore, it can be seen from the result of Table 11 that introduction of the multiple mutations listed in Table 11 increases the solubility of PheDH.

TABLE 10
Solubility of wild-type and single mutation-introduced PheDH

| | Solubility | | Solubility | | Solubility |
|---|---|---|---|---|---|
| WT | 1.8 mg/mL | K173D | 2.7 mg/mL | C256A | 5.8 mg/mL |
| R2D | 2.0 mg/mL | C200A | 10.9 mg/mL | L257K | 2.8 mg/mL |
| R2E | 3.1 mg/mL | C200S | 3.4 mg/mL | R271D | 4.8 mg/mL |
| R10D | 4.0 mg/mL | C210S | 4.6 mg/mL | R271E | 3.8 mg/mL |
| R10E | 2.7 mg/mL | C210A | 3.3 mg/mL | Q277D | 3.5 mg/mL |
| Y11E | 4.4 mg/mL | K216D | 2.9 mg/mL | K278D | 6.9 mg/mL |
| Y11D | 2.4 mg/mL | K216E | 2.7 mg/mL | K278E | 4.0 mg/mL |
| C19A | 3.8 mg/mL | K220D | 2.7 mg/mL | R279E | 2.6 mg/mL |
| C19S | 3.4 mg/mL | K220E | 2.6 mg/mL | C282S | 5.1 mg/mL |
| C44A | 7.4 mg/mL | Q222E | 7.4 mg/mL | C282A | 4.4 mg/mL |
| C44S | 4.7 mg/mL | Q222D | 7.2 mg/mL | R326E | 3.2 mg/mL |
| A50D | 3.4 mg/mL | N227D | 3.6 mg/mL | N329D | 2.8 mg/mL |
| A50E | 2.2 mg/mL | N227E | 3.2 mg/mL | N331E | 3.3 mg/mL |
| S51D | 2.8 mg/mL | R228E | 6.8 mg/mL | N331D | 3.2 mg/mL |
| S51E | 2.2 mg/mL | R228D | 6.6 mg/mL | C335A | 6.1 mg/mL |
| C70A | 3.3 mg/mL | C240S | 3.0 mg/mL | C335S | 5.2 mg/mL |
| C70S | 3.3 mg/mL | C240A | 2.9 mg/mL | R340D | 2.2 mg/mL |
| K90E | 3.1 mg/mL | R255E | 9.8 mg/mL | K348E | 2.8 mg/mL |
| K173E | 5.0 mg/mL | R255D | 8.3 mg/mL | | |

TABLE 11
Solubility of wild-type and multiple mutations-introduced PheDH

| | Solubility | | Solubility |
|---|---|---|---|
| WT | 1.8 mg/mL | Y11E/R228E/K278D | 10.4 mg/mL |
| R10D/Y11E | 4.2 mg/mL | Y11E/R228E/N290M | 3.7 mg/mL |
| R10D/K173E | 6.1 mg/mL | Y11E/R228E/N290D | 5.8 mg/mL |
| R10D/Q222D | 11.9 mg/mL | Y11E/R228E/V297F | 7.1 mg/mL |
| R10D/R228E | 5.7 mg/mL | Y11E/R255E/K278D | 10.3 mg/mL |
| R10D/R255E | 15.4 mg/mL | Y11E/R271D/K278D | 7.7 mg/mL |
| R10D/R271D | 7.3 mg/mL | Y11E/K278D/N290M | 5.0 mg/mL |
| R10D/K278D | 5.4 mg/mL | Y11E/K278D/N290D | 6.5 mg/mL |
| Y11E/K173E | 7.2 mg/mL | Y11E/K278D/V297F | 12.4 mg/mL |
| Y11E/Q222D | 3.5 mg/mL | L41W/K173E/K278D | 7.1 mg/mL |
| Y11E/R228E | 34.1 mg/mL | L41W/R228E/K278D | 8.1 mg/mL |
| Y11E/R255E | 3.4 mg/mL | K173E/K278D/N290M | 4.6 mg/mL |
| Y11E/R271D | 6.6 mg/mL | K173E/K278D/N290D | 10.5 mg/mL |
| Y11E/K278D | 12.3 mg/mL | K173E/K278D/V297F | 11.6 mg/mL |
| K173E/Q222D | 3.8 mg/mL | R228E/K278D/N290M | 6.2 mg/mL |
| K173E/R228E | 2.9 mg/mL | R228E/K278D/N290D | 9.3 mg/mL |
| K173E/R255E | 4.6 mg/mL | R228E/K278D/V297F | 7.5 mg/mL |
| K173E/R271D | 2.4 mg/mL | R10D/Y11E/K173E/N290D | 7.3 mg/mL |
| K173E/K278D | 6.8 mg/mL | R10D/Y11E/Q222D/N290D | 2.2 mg/mL |
| K173E/N290D | 7.3 mg/mL | R10D/Y11E/R228E/N290D | 3.1 mg/mL |
| Q222D/R228E | 5.7 mg/mL | R10D/Y11E/R255E/N290D | 2.4 mg/mL |
| Q222D/R255E | 4.3 mg/mL | R10D/Y11E/K278D/N290D | 2.3 mg/mL |
| Q222D/R271D | 2.6 mg/mL | R10D/T115S/Q222D/N290D | 4.3 mg/mL |
| Q222D/K278D | 3.9 mg/mL | R10D/T115S/R255E/N290D | 5.2 mg/mL |
| Q222D/N290D | 3.9 mg/mL | R10D/F124I/Q222D/N290D | 4.7 mg/mL |
| R228E/R255E | 3.4 mg/mL | R10D/F124I/R255E/N290D | 4.6 mg/mL |
| R228E/R271D | 2.3 mg/mL | R10D/K173E/Q222D/N290D | 5.7 mg/mL |
| R228E/K278D | 8.7 mg/mL | R10D/K173E/R228E/N290D | 11.7 mg/mL |
| R228E/N290D | 2.7 mg/mL | R10D/K173E/R255E/N290D | 4.7 mg/mL |
| R255E/R271D | 3.1 mg/mL | R10D/K173E/R271D/N290D | 5.0 mg/mL |
| R255E/K278D | 3.2 mg/mL | R10D/K173E/K278D/N290D | 6.9 mg/mL |
| R255E/N290D | 2.2 mg/mL | R10D/Q222D/R228E/N290D | 4.9 mg/mL |
| R271D/K278D | 4.6 mg/mL | R10D/Q222D/R255E/N290D | 4.4 mg/mL |
| R10D/Y11E/Q222D | 3.2 mg/mL | R10D/Q222D/K278D/N290D | 4.2 mg/mL |
| R10D/Y11E/R228E | 4.9 mg/mL | R10D/R228E/R255E/N290D | 5.8 mg/mL |
| R10D/Y11E/K278D | 3.1 mg/mL | R10D/R228E/K278D/N290D | 6.5 mg/mL |
| R10D/K173E/Q222D | 3.0 mg/mL | R10D/R255E/K278D/N290D | 3.8 mg/mL |
| R10D/K173E/R255E | 7.7 mg/mL | Y11E/T115S/R228E/N290D | 2.3 mg/mL |
| R10D/K173E/N290D | 4.9 mg/mL | Y11E/K173E/Q222D/N290D | 11.2 mg/mL |
| R10D/Q222D/R228E | 12.1 mg/mL | Y11E/K173E/R228E/N290D | 3.2 mg/mL |
| R10D/Q222D/R255E | 8.3 mg/mL | Y11E/K173E/R255E/N290D | 10.9 mg/mL |
| R10D/Q222D/R271D | 5.1 mg/mL | Y11E/K173E/R271D/N290D | 8.3 mg/mL |
| R10D/Q222D/K278D | 10.1 mg/mL | Y11E/K173E/K278D/N290D | 7.0 mg/mL |
| R10D/Q222D/N290M | 5.4 mg/mL | Y11E/Q222D/R228E/N290D | 15.3 mg/mL |
| R10D/Q222D/N290D | 8.6 mg/mL | Y11E/Q222D/K278D/N290D | 6.2 mg/mL |
| R10D/Q222D/V297F | 10.5 mg/mL | Y11E/R228E/R255E/N290D | 8.7 mg/mL |
| R10D/R228E/R255E | 8.7 mg/mL | Y11E/R228E/R271D/N290D | 6.5 mg/mL |
| R10D/R255E/R271D | 6.2 mg/mL | Y11E/R228E/K278D/N290D | 6.1 mg/mL |
| R10D/R255E/K278D | 11.3 mg/mL | Y11E/R255E/K278D/N290D | 16.0 mg/mL |
| R10D/R255E/N290M | 17.2 mg/mL | Y11E/R271D/K278D/N290D | 3.1 mg/mL |
| R10D/R255E/N290D | 7.7 mg/mL | T115S/K173E/K278D/N290D | 4.4 mg/mL |
| R10D/R255E/V297F | 9.8 mg/mL | T115S/R228E/K278D/N290D | 5.0 mg/mL |
| Y11E/L41W/R228E | 4.6 mg/mL | F124I/K173E/K278D/N290D | 2.0 mg/mL |
| Y11E/L41W/K278D | 11.5 mg/mL | F124I/R228E/K278D/N290D | 2.5 mg/mL |
| Y11E/K173E/R228E | 5.5 mg/mL | K173E/Q222D/K278D/N290D | 5.8 mg/mL |
| Y11E/K173E/K278D | 10.6 mg/mL | K173E/R228E/K278D/N290D | 5.1 mg/mL |
| Y11E/K173E/N290D | 5.4 mg/mL | K173E/R255E/K278D/N290D | 3.6 mg/mL |
| Y11E/Q222D/R228E | 9.4 mg/mL | Q222D/R228E/K278D/N290D | 6.8 mg/mL |
| Y11E/Q222D/K278D | 8.5 mg/mL | R228E/R255E/K278D/N290D | 6.1 mg/mL |
| Y11E/R228E/R255E | 6.6 mg/mL | R228E/R271D/K278D/N290D | 2.3 mg/mL |
| Y11E/R228E/R271D | 11.9 mg/mL | | |

### Example 6 Evaluation of Enzyme Activity of PheDH

Evaluation of the activity of each of the enzymes prepared in Example 3 was performed in accordance with the following procedure. PheDH the solvent of which had been replaced with a buffer for stock was prepared so as to be 0.1 mg/mL, and a change with the lapse of time of the absorbance at a wavelength of 340 nm of a solution obtained by adding 100 μL of 200 mM Glycine-KCl-KOH with PH 10.0, 4 μL of 50 mM NAD⁺ (manufactured by Fujifilm Wako Pure Chemical Corporation), 20 μL of a 10 mM aqueous L-phenylalanine solution, and 56 μL of ultrapure water to 20 μL of PheDH was measured with a microplate reader (SpectraMax M2e manufactured by Molecular Devices) for 5 minutes. Tables 12 and 13 list relative activity compared with the value of the wild-type PheDH as a control. The results of Tables 12 and 13 were calculated from an average value when an experience was conducted twice for the same sample.

It can be seen from the result of Table 12 that introduction of the mutations listed in Table 12 can improve the reactivity of PheDH with L-phenylalanine. Furthermore, it can be seen from the result of Table 13 that introduction of the multiple mutations listed in Table 13 can improve the reactivity of PheDH with L-phenylalanine.

TABLE 12
Activity of wild-type and single mutation-introduced PheDH

| Relative activity | | Relative activity | | Relative activity | |
|---|---|---|---|---|---|
| compared with | | compared with | | compared with | |
| control (WT) | | control (WT) | | control (WT) | |
| WT | 100% | N227D | 121% | S280D | 124% |
| C19S | 131% | N227E | 119% | C282S | 106% |
| F77L | 144% | R228E | 160% | N290C | 140% |
| F124I | 106% | R228D | 118% | N290T | 111% |
| T147S | 160% | C234A | 131% | L294Q | 111% |
| K173E | 122% | C234S | 123% | Q296D | 134% |
| K173D | 106% | C240S | 164% | Q296E | 127% |
| C200A | 115% | C240A | 140% | V297T | 128% |
| C200S | 113% | R255E | 135% | V297G | 111% |
| K216D | 160% | R255D | 126% | K328E | 114% |
| K216E | 122% | C256A | 109% | K328D | 105% |
| K220E | 152% | R271E | 107% | N329D | 117% |
| K220D | 136% | K278D | 139% | N331D | 122% |
| Q222D | 152% | R279D | 111% | R340E | 108% |
| Q222E | 127% | R279E | 108% | K347D | 109% |

TABLE 13
Activity of wild-type and multiple mutations-introduced PheDH

| Relative activity compared with | | Relative activity | |
|---|---|---|---|
| control (WT) | | compared with control (WT) | |
| WT | 100% | R10D/Q222D/R255E | 106% |
| K173E/Q222D | 110% | R10D/Q222D/R271D | 113% |
| C210S/N290D | 120% | R10D/R228E/R255E | 127% |
| Q222D/R228E | 112% | R10D/R255E/R271D | 124% |
| R228E/R255E | 111% | Y11E/K173E/R228E | 111% |
| N290D/C335A | 122% | Y11E/K173E/N290D | 116% |
| R10D/Y11E/Q222D | 121% | Y11E/R228E/N290D | 112% |
| R10D/Y11E/R255E | 115% | Y11E/K278D/N290D | 122% |
| R10D/Y11E/K278D | 108% | R10D/Y11E/R255E/N290D | 111% |
| R10D/K173E/Q222D | 129% | R10D/R228E/R255E/N290D | 144% |
| R10D/K173E/R255E | 118% | Y11E/K173E/R255E/N290D | 166% |
| R10D/Q222D/R228E | 124% | Y11E/R255E/K278D/N290D | 119% |

### INDUSTRIAL APPLICABILITY

The modified phenylalanine dehydrogenase is useful for quick, high-precision, and highly sensitive measurement of phenylalanine and/or production of phenylpyruvate. The modified phenylalanine dehydrogenase is also useful as a liquid reagent. The modified phenylalanine dehydrogenase is useful as a liquid reagent in particular. The method of analysis is useful for diagnosis of diseases such as phenylketonuria and measurement of a phenylalanine content in food, for example.

### SEQUENCE LISTING FREE TEXT

SEQ ID NO:1 indicates the amino acid sequence of the Thermoactinomyces intermedius phenylalanine dehydrogenase (PheDH).

SEQ ID NO:2 indicates the codon-optimized nucleotide sequence encoding the amino acid sequence of the Thermoactinomyces intermedius PheDH (SEQ ID NO:1).

SEQ ID NOS:3 to 6 indicate the amino acid sequences of the respective motifs in PheDH.

SEQ ID NOS:7 to 9 indicate the amino acid sequences of the respective motifs indicated by the respective shorter amino acid sequences in PheDH.

SEQ ID NOS:10 to 12 indicate amino acid sequences near the respective motifs in the Thermoactinomyces intermedius PheDH.

SEQ ID NOS:13 to 15 indicate consensus amino acid sequences (amino acid sequences having a high degree of commonness) near the respective motifs in PheDH.

SEQ ID NOS:16, 26, 35, 44, 54, 62, 70, 79, 88, 97, and 106 indicate the amino acid sequences of PheDH derived from the respective species.

SEQ ID NOS:17 to 22, 27 to 31, 36 to 40, 45 to 50, 55 to 58, 63 to 66, 71 to 75, 80 to 84, 89 to 93, 98 to 102, and 107 indicate the amino acid sequences of conservations regions corresponding to the respective motifs in PheDH derived from the respective species.

SEQ ID NOS:23 to 25, 32 to 34, 41 to 43, 51 to 53, 59 to 61, 67 to 69, 76 to 78, 85 to 87, 94 to 96, 103 to 105, and 108 to 110 indicate amino acid sequences near the respective motifs in PheDH derived from the respective species.

SEQ ID NO:111 indicates the amino acid sequence of Thermoactinomyces intermedius PheDH in which the His-tag is added to the N-terminus.

SEQ ID NO:112 indicates a codon-optimized nucleotide sequence encoding the amino acid sequence of Thermoactinomyces intermedius PheDH in which the His-tag is added to the N-terminus (SEQ ID NO:111).

SEQ ID NO:113 indicates a linker nucleotide sequence (an NdeI site+an initiation codon+a His-tag code sequence) added to the 5′-end of the nucleotide sequence of SEQ ID NO:2 in order to form a DNA fragment including the nucleotide sequence of SEQ ID NO:112.

SEQ ID NO:114 indicates a linker nucleotide sequence (a termination codon+a BamHI site) added to the 3′-end of the nucleotide sequence of SEQ ID NO:2 in order to form the DNA fragment including the nucleotide sequence of SEQ ID NO:112.

1. A modified phenylalanine dehydrogenase comprising a mutation of at least one amino acid residue in a motif selected from the group consisting of:
(1)
(SEQ ID NO: 3)
GPALGGXRM,
(2)
(SEQ ID NO: 4)
GRFXTGTDMGT,
(3)
DF motif,
(4)
(SEQ ID NO: 5
GXANN,
(5)
RH,
(6)
(SEQ ID NO: 6)
VNXGGLIQV,
and
combinations thereof;
wherein X is any amino acid,
wherein said modified phenylalanine dehydrogenase comprises at least one motif selected from the group consisting of the motifs (1) to (6),
wherein said modified phenylalanine dehydrogenase has a phenylalanine dehydrogenase activity, and
wherein said modified phenylalanine dehydrogenase has at least one characteristic selected from the group consisting of substrate specificity, solubility, and a phenylalanine dehydrogenase activity that is higher than a non-modified phenylalanine dehydrogenase.

2. The modified phenylalanine dehydrogenase according to claim 1, wherein the mutation is a substitution selected from the group consisting of:
(a) a substitution of leucine in
(SEQ ID NO: 3)
GPALGGXRM;
(b) a substitution of the seventh amino acid
threonine in
(SEQ ID NO: 4)
GRFXTGTDMGT;
(c) a substitution of phenylalanine in
DF;
(d) a substitution of the fourth amino acid
asparagine in
(SEQ ID NO: 5)
GXANN;
(e) a substitution of arginine in
RH;
(f) a substitution of asparagine in
(SEQ ID NO: 6)
VNXGGLIQV;
(g) a substitution of leucine in
(SEQ ID NO: 6)
VNXGGLIQV;
(h) a substitution of glutamine in
(SEQ ID NO: 6)
VNXGGLIQV;
(i) a substitution of the ninth amino acid
valine in
(SEQ ID NO: 6)
VNXGGLIQV;
and
(j) combinations thereof.

3. The modified phenylalanine dehydrogenase according to claim 2, wherein the mutation is a substitution selected from the group consisting of:
(a) a substitution of leucine in GPALGGXRM (SEQ ID NO:3) with tryptophane, phenylalanine, tyrosine, or methionine;
(b) a substitution of the seventh amino acid threonine in GRFXTGTDMGT (SEQ ID NO:4) with serine;
(c) a substitution of phenylalanine in DF with leucine or isoleucine;
(d) a substitution of the fourth amino acid asparagine in GXANN (SEQ ID NO:5) with glycine, glutamine, threonine, lysine, proline, or serine;
(e) a substitution of arginine in RH with aspartic acid or glutamic acid;
(f) a substitution of asparagine in VNXGGLIQV (SEQ ID NO:6) with valine, aspartic acid, methionine, glutamine, proline, isoleucine, histidine, alanine, threonine, glycine, or cysteine;
(g) a substitution of leucine in VNXGGLIQV (SEQ ID NO:6) with phenylalanine, glutamine, histidine, asparagine, isoleucine, aspartic acid, glycine, glutamic acid, threonine, or serine;
(h) a substitution of glutamine in VNXGGLIQV (SEQ ID NO:6) with aspartic acid, glutamic acid, lysine, asparagine, serine, or arginine; and
(i) a substitution of the ninth amino acid valine in VNXGGLIQV (SEQ ID NO:6) with tyrosine, tryptophane, glutamic acid, asparagine, threonine, isoleucine, lysine, glycine, serine, leucine, methionine, glutamine, phenylalanine, cysteine, or arginine.

4. The modified phenylalanine dehydrogenase according to claim 1, wherein the phenylalanine dehydrogenase comprises the motifs (1) to (6) in numerical order.

5. The modified phenylalanine dehydrogenase according to claim 1, wherein the phenylalanine dehydrogenase is derived from the genus Thermoactinomyces.

6. The modified phenylalanine dehydrogenase according to claim 1, wherein the phenylalanine dehydrogenase comprises the following:
(A) the amino acid sequence of SEQ ID NO:1;
(B) an amino acid sequence comprising a substitution, deletion, insertion, or addition of one or several amino acid residues in the amino acid sequence of SEQ ID NO:1; or
(C) an amino acid sequence having 90% or more identity to the amino acid sequence of SEQ ID NO:1.

7. A modified phenylalanine dehydrogenase,
comprising one or more mutations of an amino acid residue selected from the group consisting of:
R2, R10, Y11, C19, L41, G42, G43, C44, A50, S51, M66, C70, F77, K90, Y112, T115, D116, F124, R129, L137, K139, S140, K144, T147, K173, C200, C210, K216, K220, Q222, N227, R228, C234, C240, R255, C256, L257, N264, R271, Q277, K278, R279, S280, C282, N290, G293, L294, Q296, V297, R326, K328, N329, N331, C335, R340, K347, K348, and combinations thereof;
wherein said phenylalanine dehydrogenase comprises:
(A) the amino acid sequence of SEQ ID NO:1,
(B) an amino acid sequence comprising substitution, deletion, insertion, or addition of one or several amino acid residues in the amino acid sequence of SEQ ID NO:1, or
(C) an amino acid sequence having 90% or more identity to the amino acid sequence of SEQ ID NO:1,
wherein said modified phenylalanine dehydrogenase has a phenylalanine dehydrogenase activity, and
wherein said modified phenylalanine dehydrogenase has an improved characteristic selected from the group consisting of substrate specificity, solubility, phenylalanine dehydrogenase activity, and combinations thereof.

8. The modified phenylalanine dehydrogenase according to claim 7, which comprises one or more substitutions of an amino acid residue selected from the following:
R2D, R2E, R10D, R10E, Y11E, Y11D, C19A, C19S, L41W, L41F, L41Y, L41M, G42A, G43A, C44A, C44S, A50D, A50E, S51D, S51E, M66I, M66L, M66V, C70A, C70S, F77L, F77I, F77R, K90E, Y112L, T115S, D116E, F124L, F124I, R129K, L137V, K139E, S140A, K144G, T147A, T147S, T147N, K173E, K173D, C200A, C200S, C210S, C210A, K216D, K216E, K220D, K220E, Q222E, Q222D, N227D, N227E, R228E, R228D, C234A, C234S, C240S, C240A, R255E, R255D, C256A, C256S, L257K, N264G, N264Q, N264T, N264K, N264P, N264S, R271D, R271E, Q277D, K278D, K278E, R279D, R279E, S280D, C282S, C282A, N290V, N290D, N290M, N290Q, N290P, N290I, N290H, N290A, N290T, N290G, N290C, G293A, L294F, L294Q, L294H, L294N, L294I, L294D, L294G, L294E, L294T, L294S, Q296D, Q296E, Q296K, Q296N, Q296S, Q296R, V297Y, V297W, V297E, V297N, V297T, V297I, V297K, V297G, V297S, V297L, V297M, V297Q, V297F, V297C, V297R, R326E, K328E, K328D, N329D, N331E, N331D, C335A, C335S, R340D, R340E, K347D, K348E, and combinations thereof.

9. A method for analyzing phenylalanine, the method comprising measuring phenylalanine contained in a test sample using the modified phenylalanine dehydrogenase according to claim 1.

10. The method according to claim 9, comprising mixing the test sample with nicotinamide adenine dinucleotide (NAD⁺) and detecting NADH produced from NAD⁺ by an action of the modified phenylalanine dehydrogenase.

11. A method for producing phenylpyruvate, the method comprising producing phenylpyruvate from phenylalanine using the modified phenylalanine dehydrogenase according to claim 1.

12. A polynucleotide encoding the modified phenylalanine dehydrogenase according to claim 1.

13. An expression vector comprising the polynucleotide according to claim 12.

14. A transformant comprising an expression unit of a polynucleotide encoding the modified phenylalanine dehydrogenase according to claim 1.

15. A method for producing a modified phenylalanine dehydrogenase, the method comprising producing a modified phenylalanine dehydrogenase comprising a mutation of at least one amino acid residue so as to improve a characteristic selected from the group consisting of a substrate specificity, a solubility, a phenylalanine dehydrogenase activity, and combinations thereof, using the transformant according to claim 14.

16. A kit for analyzing phenylalanine, the kit comprising the modified phenylalanine dehydrogenase according to claim 1.

17. The kit for analyzing phenylalanine according to claim 16, further comprising at least one of a buffer solution or a buffer salt for reaction and nicotinamide adenine dinucleotide (NAD⁺).

18. An enzyme sensor for analyzing phenylalanine, the enzyme sensor comprising (a) an electrode for detection and (b) the modified phenylalanine dehydrogenase according to claim 1 immobilized or disposed on the electrode for detection.


SEQUENCE LISTING (114 sequences)

SEQ ID NO: 1 - protein, 366 aa, Thermoactinomyces intermedius
MRDVFEMMDRYGHEQVIFCRHPQTGLKAIIALHNTTAGPALGGCRMIPYASTDEALEDVLRLSKGMTYKCSLADVDFGGGKMVIIGDPKKDKSPELFRVIGRFVGGLNGRFYTGTDMGTNPEDFVHAARESKSFAGLPKSYGGKGDTSIPTALGVFHGMRATARFLWGTDQLKGRVVAIQGVGKVGERLLQLLVEVGAYCKIADIDSVRCEQLKEKYGDKVQLVDVNRIHKESCDIFSPCAKGGVVNDDTIDEFRCLAIVGSANNQLVEDRHGALLQKRSICYAPDYLVNAGGLIQVADELEGFHEERVLAKTEAIYDMVLDIFHRAKNENITTCEAADRIVMERLKKLTDIRRILLEDPRNSARR

SEQ ID NO: 2 - DNA, 1101 bp, Thermoactinomyces intermedius
atgcgcgacgtgttcgaaatgatggatcgttatggccatgaacaggttatcttttgtcgccatccacaaacgggcttgaaagccattatcgccttacacaacacgacagcgggtccagctctgggtggttgccgtatgatcccgtatgcgtcaaccgatgaagcactcgaagatgttctgcgcctgtctaaaggtatgacctacaaatgctctctggcagatgtcgatttcggcggtggcaaaatggtcatcattggagatccgaagaaagacaaaagtccggaactgtttcgcgtcattggtcgcttcgtgggaggcttaaacggccggttttataccggtaccgatatggggactaaccctgaggactttgtgcatgcggcgcgtgaatcgaaaagctttgcaggactgcccaaatcctatggcggtaaaggcgatacctccatcccgacagccctgggcgtatttcatgggatgcgtgccactgcccgctttctctggggcacggatcagcttaaaggtcgggttgtcgcgattcaaggggttggcaaagttggcgaacgcctgttgcagttactcgtcgaggtaggcgcttactgcaagattgcggatatcgatagcgttcgttgtgagcaactgaaagagaaatacggggataaagtgcagttagtggacgtaaaccgcattcacaaggaaagctgcgatatcttttcgccttgtgctaaaggaggggtggtcaatgatgatacgattgacgagttccgctgcttggcgattgtgggctcagccaacaatcagctggtggaggatcgtcatggtgcgcttttgcagaaacgtagcatctgctatgcgccggactatctggtgaatgctggtggcctgatccaggttgcggacgaactggaaggtttccatgaagaacgtgtgctcgccaaaacggaagctatttacgacatggtactggacattttccatcgtgccaagaacgagaatatcaccacctgtgaagcagcagatcgcattgtgatggaacgcctgaagaaactgactgacattcggcgcattcttctggaagatccgcgcaatagtgcacgccgttaa

SEQ ID NO: 3 - protein, 9 aa, Artificial Sequence
  motif
  misc_feature (7)..(7): Xaa can be any naturally occurring amino acid
GPALGGXRM

SEQ ID NO: 4 - protein, 11 aa, Artificial Sequence
  motif
  misc_feature (4)..(4): Xaa can be any naturally occurring amino acid
GRFXTGTDMGT

SEQ ID NO: 5 - protein, 5 aa, Artificial Sequence
  motif
  misc_feature (2)..(2): Xaa can be any naturally occurring amino acid
GXANN

SEQ ID NO: 6 - protein, 9 aa, Artificial Sequence
  motif
  misc_feature (3)..(3): Xaa can be any naturally occurring amino acid
VNXGGLIQV

SEQ ID NO: 7 - protein, 6 aa, Artificial Sequence
  motif
GPALGG

SEQ ID NO: 8 - protein, 7 aa, Artificial Sequence
  motif
TGTDMGT

SEQ ID NO: 9 - protein, 6 aa, Artificial Sequence
  motif
GGLIQV

SEQ ID NO: 10 - protein, 33 aa, Thermoactinomyces intermedius
LHNTTAGPALGGCRMIPYASTDEALEDVLRLSK

SEQ ID NO: 11 - protein, 55 aa, Thermoactinomyces intermedius
GGLNGRFYTGTDMGTNPEDFVHAARESKSFAGLPKSYGGKGDTSIPTALGVFHGM

SEQ ID NO: 12 - protein, 57 aa, Thermoactinomyces intermedius
DTIDEFRCLAIVGSANNQLVEDRHGALLQKRSICYAPDYLVNAGGLIQVADELEGFH

SEQ ID NO: 13 - protein, 31 aa, Artificial Sequence
  consensus sequence
HDTTLGPALGGCRMIPYSTDEALEDVLRLSK

SEQ ID NO: 14 - protein, 53 aa, Artificial Sequence
  consensus sequence
ESLNGRFYTGTDMGTTPEDFVHAAKETNIVGLPEEYGGSGDSSIPTALGVYGL

SEQ ID NO: 15 - protein, 56 aa, Artificial Sequence
  consensus sequence
ETIQLKVKAIGSANNQLLNEDRHGDLLEKGILYAPDYIVNAGGLIQLVADELYGPN

SEQ ID NO: 16 - protein, 386 aa, Lihuaxuella thermophile
MEGMKTEHVRTDIREEKQTMMRDIFELMDQYGHEQVVFCRHPRSGLKAIIAIHNTTMGPALGGCRMIPYSSTDEALEDVLRLSKGMTYKCGVADVDFGGGKMVIIGDPERDKSPEMFRAVGRFVEGLNGRFFTGTDMGTNPEDFVYAARESRSFVGLPAAHGGSGDTSIPTALGVLQGLKATAKHLWGTTDLHGRTIAIQGVGKVGGRLLELLMEEGAHCIIADLNESKVRQYKEKYKEQVELSEVNIIHRSDCDIFSPCARGGVINDQTLPELRCQAIVGSANNQLVEDRHGDLLHEKGILYAPDYLVNAGGLIQVADELEGFSEERVLAKTKAIYDMLLQIYELSEKEQIPTCRAADRLVVERLEKVADLRRILLGWGRDARGR

SEQ ID NO: 17 - protein, 12 aa, Lihuaxuella thermophile
GPALGGCRMIPY

SEQ ID NO: 18 - protein, 13 aa, Lihuaxuella thermophile
TGTDMGTNPEDFV

SEQ ID NO: 19 - protein, 13 aa, Lihuaxuella thermophile
TGTDMGTNPEDFV

SEQ ID NO: 20 - protein, 16 aa, Lihuaxuella thermophile
AIVGSANNQLVEDRHG

SEQ ID NO: 21 - protein, 16 aa, Lihuaxuella thermophile
AIVGSANNQLVEDRHG

SEQ ID NO: 22 - protein, 22 aa, Lihuaxuella thermophile
YAPDYLVNAGGLIQVADELEGF

SEQ ID NO: 23 - protein, 33 aa, Lihuaxuella thermophile
IHNTTMGPALGGCRMIPYSSTDEALEDVLRLSK

SEQ ID NO: 24 - protein, 55 aa, Lihuaxuella thermophile
EGLNGRFFTGTDMGTNPEDFVYAARESRSFVGLPAAHGGSGDTSIPTALGVLQGL

SEQ ID NO: 25 - protein, 57 aa, Lihuaxuella thermophile
QTLPELRCQAIVGSANNQLVEDRHGDLLHEKGILYAPDYLVNAGGLIQVADELEGFS

SEQ ID NO: 26 - protein, 368 aa, Baia soyae
MEENRNEYVMKDIFACMEEGGHEQVTFFRHEKSGLRAIVAIHDSTLGPALGGCRMIPYASTQEALDDVLQLSQGMTHKCAIADVDFGGGKMVILPDSSSTEKSPELFRAVGRFVGSLQGRFFTGTDMGTSPEDFIHAAKESKYIVGLPEYYGGSGDTSISTAMGVLQGMRATAQYMYGSESLEGKVVAIQGIGKVGARLAGLLLAEGAELIIADVHLPSCLIIQRMSPESVEITTTHKILETPCDFLVPCARGGIVNTSNIHQFRCKAIIGSANNQLENDQCADLLAEREILYAPDYLVNAGGLIQVAMEILQKTKECAEEKARFIYDMLLQVYKRAEQHQISTCQAANEIVEERLEKIADIRRIYLG

SEQ ID NO: 27 - protein, 12 aa, Baia soyae
GPALGGCRMIPY

SEQ ID NO: 28 - protein, 7 aa, Baia soyae
TGTDMGT

SEQ ID NO: 29 - protein, 4 aa, Baia soyae
PEDF

SEQ ID NO: 30 - protein, 7 aa, Baia soyae
GSANNQL

SEQ ID NO: 31 - protein, 16 aa, Baia soyae
YAPDYLVNAGGLIQVA

SEQ ID NO: 32 - protein, 33 aa, Baia soyae
IHDSTLGPALGGCRMIPYASTQEALDDVLQLSQ

SEQ ID NO: 33 - protein, 55 aa, Baia soyae
GSLQGRFFTGTDMGTSPEDFIHAAKESKYIVGLPEYYGGSGDTSISTAMGVLQGM

SEQ ID NO: 34 - protein, 57 aa, Baia soyae
SNIHQFRCKAIIGSANNQLENDQCADLLAEREILYAPDYLVNAGGLIQVAMEILQKT

SEQ ID NO: 35 - protein, 368 aa, Thermoactinomyces sp.
MEENRTEYVMKDIFDCMEEGGHEQVTFFRHDKSGLRAIVAIHDSTLGPALGGCRMIPYSSTQEALDDVLQLSRGMTHKCAIADVDFGGGKMVILPDSSHTEKSPELFRAVGRFVGSLQGRFYTGTDMGTTPEDFIHAAKESKYIVGLPEYYGGSGDTSISTAMGVLQGMRATAEYLYGSESLEGKVIAIQGIGKVGARLAGLLLAEGAELIIADVHLPSCLIIQRMSPESVEITTTHKILETPCDFLVPCARGGIVNTSNIHQFRCRAIIGSANNQLENDECADLLAQREILYAPDYLVNAGGLIQVAMEILQKTKECAEEKARFIYEMLLQVYKRAEQHQISTCQAANEIVEERLEKIADIRRIYLG

SEQ ID NO: 36 - protein, 12 aa, Thermoactinomyces sp.
GPALGGCRMIPY

SEQ ID NO: 37 - protein, 11 aa, Thermoactinomyces sp.
GRFYTGTDMGT

SEQ ID NO: 38 - protein, 4 aa, Thermoactinomyces sp.
PEDF

SEQ ID NO: 39 - protein, 7 aa, Thermoactinomyces sp.
GSANNQL

SEQ ID NO: 40 - protein, 16 aa, Thermoactinomyces sp.
YAPDYLVNAGGLIQVA

SEQ ID NO: 41 - protein, 33 aa, Thermoactinomyces sp.
IHDSTLGPALGGCRMIPYSSTQEALDDVLQLSR

SEQ ID NO: 42 - protein, 55 aa, Thermoactinomyces sp.
GSLQGRFYTGTDMGTTPEDFIHAAKESKYIVGLPEYYGGSGDTSISTAMGVLQGM

SEQ ID NO: 43 - protein, 57 aa, Thermoactinomyces sp.
SNIHQFRCRAIIGSANNQLENDECADLLAQREILYAPDYLVNAGGLIQVAMEILQKT

SEQ ID NO: 44 - protein, 370 aa, Caldalkalibacillus thermarum
MSTVTFDQISEHEQVMFCNDPHTGLKAIIAIHNTTLGPALGGCRMLPYKSEEEALTDVLRLSKGMTYKCVAADVDFGGGKAVIIGDPRKDKTPELFRAFGQFVQSLNGRFYTGTDMGTTPEDFVQAYKETSFIVGLPEEYGGNGDSSVTTAFGVMQGLRAVSQFLWGTDVLTERVFAVQGLGKVGFKVAEGLLKEGANVYVTDVDPETIAKLEEKAYQYPGHVQAVTADDIYGVGADVFVPCAIGGIINDETIERLKVKAVCGAANNQLLEDRHGKVLQAKNILYAPDYIVNAGGLIQVSDELYGPNKARVLKKTRALYDTLFEIFQSAEKKAVSTVEAANQFVEERLQKRARLNSFFSPDNPPKWRVRR

SEQ ID NO: 45 - protein, 9 aa, Caldalkalibacillus thermarum
GPALGGCRM

SEQ ID NO: 46 - protein, 13 aa, Caldalkalibacillus thermarum
LNGRFYTGTDMGT

SEQ ID NO: 47 - protein, 5 aa, Caldalkalibacillus thermarum
PEDFV

SEQ ID NO: 48 - protein, 5 aa, Caldalkalibacillus thermarum
ANNQL

SEQ ID NO: 49 - protein, 5 aa, Caldalkalibacillus thermarum
EDRHG

SEQ ID NO: 50 - protein, 9 aa, Caldalkalibacillus thermarum
VNAGGLIQV

SEQ ID NO: 51 - protein, 33 aa, Caldalkalibacillus thermarum
IHNTTLGPALGGCRMLPYKSEEEALTDVLRLSK

SEQ ID NO: 52 - protein, 55 aa, Caldalkalibacillus thermarum
QSLNGRFYTGTDMGTTPEDFVQAYKETSFIVGLPEEYGGNGDSSVTTAFGVMQGL

SEQ ID NO: 53 - protein, 57 aa, Caldalkalibacillus thermarum
ETIERLKVKAVCGAANNQLLEDRHGKVLQAKNILYAPDYIVNAGGLIQVSDELYGPN

SEQ ID NO: 54 - protein, 380 aa, Bacillus badius
MSLVEKTSIIKDFTLFEKMSEHEQVVFCNDPATGLRAIIAIHDTTLGPALGGCRMQPYNSVEEALEDALRLSKGMTYKCAASDVDFGGGKAVIIGDPQKDKSPELFRAFGQFVDSLGGRFYTGTDMGTNMEDFIHAMKETNCIVGVPEAYGGGGDSSIPTAMGVLYGIKATNKMLFGKDDLGGVTYAIQGLGKVGYKVAEGLLEEGAHLFVTDINEQTLEAIQEKAKTTSGSVTVVASDEIYSQEADVFVPCAFGGVVNDETMKQFKVKAIAGSANNQLLTEDHGRHLADKGILYAPDYIVNSGGLIQVADELYEVNKERVLAKTKHIYDAILEVYQQAELDQITTMEAANRMCEQRMAARGRRNSFFTSSVKPKWDIRN

SEQ ID NO: 55 - protein, 9 aa, Bacillus badius
GPALGGCRM

SEQ ID NO: 56 - protein, 12 aa, Bacillus badius
GRFYTGTDMGTN

SEQ ID NO: 57 - protein, 7 aa, Bacillus badius
GSANNQL

SEQ ID NO: 58 - protein, 10 aa, Bacillus badius
GGLIQVADEL

SEQ ID NO: 59 - protein, 33 aa, Bacillus badius
IHDTTLGPALGGCRMQPYNSVEEALEDALRLSK

SEQ ID NO: 60 - protein, 55 aa, Bacillus badius
DSLGGRFYTGTDMGTNMEDFIHAMKETNCIVGVPEAYGGGGDSSIPTAMGVLYGI

SEQ ID NO: 61 - protein, 57 aa, Bacillus badius
ETMKQFKVKAIAGSANNQLLTEDHGRHLADKGILYAPDYIVNSGGLIQVADELYEVN

SEQ ID NO: 62 - protein, 366 aa, Fictibacillus nanhaiensis
MFEKISQHEQVVFCNDPSTGLKAIIAIHNTTLGPALGGCRMRPYGSVDEALEDVLRLSKGMTYKCAGADVDFGGGKSVIIGDPMTDRTPELFRAFGQFVDSLNGRFYTGTDMGTTPDDFMHALKETNCIVGVPEEYGGSGDSSVPTAQGVIYGLQATIQTLEGTDELSGKSYSIQGLGKVGFKVAEQLLAAGTQIYVTDINEKALKMIQERAELLPGNVEVVEGSDIYGVDADIFIPCALGGIIHDETIEQLKVKAIVGSANNQLLEDKHGLYLQQKGILYGPDYIVNAGGLIQVADELYGPNKARVLTKTRAIYDSLIQIYSESTKNQISTMEAANLFCEEKLLARSKRNSFFAHNRRPKWQVRH

SEQ ID NO: 63 - protein, 9 aa, Fictibacillus nanhaiensis
GPALGGCRM

SEQ ID NO: 64 - protein, 13 aa, Fictibacillus nanhaiensis
LNGRFYTGTDMGT

SEQ ID NO: 65 - protein, 10 aa, Fictibacillus nanhaiensis
AIVGSANNQL

SEQ ID NO: 66 - protein, 13 aa, Fictibacillus nanhaiensis
VNAGGLIQVADEL

SEQ ID NO: 67 - protein, 33 aa, Fictibacillus nanhaiensis
IHNTTLGPALGGCRMRPYGSVDEALEDVLRLSK

SEQ ID NO: 68 - protein, 55 aa, Fictibacillus nanhaiensis
DSLNGRFYTGTDMGTTPDDFMHALKETNCIVGVPEEYGGSGDSSVPTAQGVIYGL

SEQ ID NO: 69 - protein, 57 aa, Fictibacillus nanhaiensis
ETIEQLKVKAIVGSANNQLLEDKHGLYLQQKGILYGPDYIVNAGGLIQVADELYGPN

SEQ ID NO: 70 - protein, 369 aa, Bacillus sp.
MDIFTKAYEHEQIVFCNDPDSGLRAIIAIHNTTLGPALGGCRMLPYASTEEALEDVLRLSKGMTYKCAAADVDFGGGKSVIIGDPNKDRSPQLFRAFGQFVESLNGRFYTGTDMGTTPEDFVHAAKETNCIVGIPEEYGGSGDSSVPTALGVIYGIKASNKVAFGDEKLEGKTYSIQGLGKVGFKVATQLLENGANLYVTDINEKAIQDLVELSKQYDGTVKVVSSDEIYSVDADVFVPCALGAIINDDTIQQLKVKAIAGSANNQLLNEERHGQELYEKGILYAPDYIVNAGGLIQVADELYGPNKNRVLKKTEAIYSSLLEVYKYSESEKIPTYKAANLFVEKRIEERKKRNSFFTHHKRPKWDIKK

SEQ ID NO: 71 - protein, 9 aa, Bacillus sp.
GPALGGCRM

SEQ ID NO: 72 - protein, 13 aa, Bacillus sp.
LNGRFYTGTDMGT

SEQ ID NO: 73 - protein, 8 aa, Bacillus sp.
PEDFVHAA

SEQ ID NO: 74 - protein, 7 aa, Bacillus sp.
GSANNQL

SEQ ID NO: 75 - protein, 13 aa, Bacillus sp.
VNAGGLIQVADEL

SEQ ID NO: 76 - protein, 33 aa, Bacillus sp.
IHNTTLGPALGGCRMLPYASTEEALEDVLRLSK

SEQ ID NO: 77 - protein, 55 aa, Bacillus sp.
ESLNGRFYTGTDMGTTPEDFVHAAKETNCIVGIPEEYGGSGDSSVPTALGVIYGI

SEQ ID NO: 78 - protein, 58 aa, Bacillus sp.
DTIQQLKVKAIAGSANNQLLNEERHGQELYEKGILYAPDYIVNAGGLIQVADELYGPN

SEQ ID NO: 79 - protein, 381 aa, Bacillus sphaericus
MAKQLEKSSKIGNEDVFQKIANHEQIVFCNDPVSGLQAIIAIHDTTLGPALGGTRMYPYKNVDEALEDVLRLSEGMTYKCAAADIDFGGGKAVIIGDPEKDKSPALFRAFGQFVESLNGRFYTGTDMGTTMDDFVHAQKETNFINGIPEQYGGSGDSSIPTAQGVIYALKATNQYLFGSDSLSGKTYAIQGLGKVGYKVAEQLLKAGADLFVTDIHENVLNSIKQKSEELGGSVTIVKSDDIYSVQADIFVPCAMGGIINDKTIPKLKVKAVVGSANNQLKDLRHANVLNEKGILYAPDYIVNAGGLIQVADELYGPNKERVLLKTKEIYRSLLEIFNQAALDCITTVEAANRKCQKTIEGQQTRNSFFSRGRRPKWNIKE

SEQ ID NO: 80 - protein, 6 aa, Bacillus sphaericus
GPALGG

SEQ ID NO: 81 - protein, 13 aa, Bacillus sphaericus
LNGRFYTGTDMGT

SEQ ID NO: 82 - protein, 5 aa, Bacillus sphaericus
DFVHA

SEQ ID NO: 83 - protein, 8 aa, Bacillus sphaericus
VGSANNQL

SEQ ID NO: 84 - protein, 13 aa, Bacillus sphaericus
VNAGGLIQVADEL

SEQ ID NO: 85 - protein, 33 aa, Bacillus sphaericus
IHDTTLGPALGGTRMYPYKNVDEALEDVLRLSE

SEQ ID NO: 86 - protein, 55 aa, Bacillus sphaericus
ESLNGRFYTGTDMGTTMDDFVHAQKETNFINGIPEQYGGSGDSSIPTAQGVIYAL

SEQ ID NO: 87 - protein, 57 aa, Bacillus sphaericus
KTIPKLKVKAVVGSANNQLKDLRHANVLNEKGILYAPDYIVNAGGLIQVADELYGPN

SEQ ID NO: 88 - protein, 379 aa, Sporosarcina ureae
MILVTLEQTLQDDKASVLDKMVEHEQILFCHDKATGLQAIIAVHDTTMGPALGGCRMAPYKTMDLALKDVLRLSKGMTYKCAAADVDFGGGKSVIIGDPLKDKTPEKFRAFGQFIESLNGRFYTGTDMGTTLEDFVHAMKETNYIVGKPVEYGGGGDSSIPTALGVFYGIKATNQNLFGDDKVEGRKYSIQGLGKVGYKVAEHIINEGGNVIVTDINEQAIADIQKLGGSAVRVVSSEEIYSQQADVFVPCAFGGVINDDTLKVLKVRGISGSANNQLAESRHGELLREKGILYAPDYIVNGGGLIQVADELYGTNPARVLAKTENIYTSLLEVFHQAEQDHMTTATAADRMCEKRIADAKNRNSFFTQSNRPKWNFHQ

SEQ ID NO: 89 - protein, 9 aa, Sporosarcina ureae
GPALGGCRM

SEQ ID NO: 90 - protein, 13 aa, Sporosarcina ureae
LNGRFYTGTDMGT

SEQ ID NO: 91 - protein, 6 aa, Sporosarcina ureae
EDFVHA

SEQ ID NO: 92 - protein, 7 aa, Sporosarcina ureae
GSANNQL

SEQ ID NO: 93 - protein, 10 aa, Sporosarcina ureae
GGLIQVADEL

SEQ ID NO: 94 - protein, 33 aa, Sporosarcina ureae
VHDTTMGPALGGCRMAPYKTMDLALKDVLRLSK

SEQ ID NO: 95 - protein, 55 aa, Sporosarcina ureae
ESLNGRFYTGTDMGTTLEDFVHAMKETNYIVGKPVEYGGGGDSSIPTALGVFYGI

SEQ ID NO: 96 - protein, 57 aa, Sporosarcina ureae
DTLKVLKVRGISGSANNQLAESRHGELLREKGILYAPDYIVNGGGLIQVADELYGTN

SEQ ID NO: 97 - protein, 379 aa, Bacillus halodurans
MLTKTPTVTSTLDIFTEMAEHEQVLFCHDPSSGLRAIIAIHDTTLGPALGGCRMYPYQTTEDALRDVLRLSKGMTQKCAAADVDFGGGKAVIIGDPAKDKSANLFRAFGQFVESLNGRFYTGTDMGTTMEDFVHALKETNGIVGIPKEYGGSGDSSVPTAKGVINSLKAISQVVLKDKQFSGRTYAIQGLGKVGFKVAEELLKEGNDLYVSDLQESLPLRLQQLGQRLGRHVEILHGDEIYEAAADVFVPCAQGAILNDATIARLKVKAIAGAANNQLEAERHGQMLHDQGIWFAPDYIVNSGGLIQVADELYGSNEKRVLSKTNAIYDTILEIFHQAERHHITTLQAANQLCERRIRERARRNNFFVNRIRPKWNLRK

SEQ ID NO: 98 - protein, 9 aa, Bacillus halodurans
GPALGGCRM

SEQ ID NO: 99 - protein, 13 aa, Bacillus halodurans
LNGRFYTGTDMGT

SEQ ID NO: 100 - protein, 6 aa, Bacillus halodurans
EDFVHA

SEQ ID NO: 101 - protein, 5 aa, Bacillus halodurans
ANNQL

SEQ ID NO: 102 - protein, 10 aa, Bacillus halodurans
GGLIQVADEL

SEQ ID NO: 103 - protein, 33 aa, Bacillus halodurans
IHDTTLGPALGGCRMYPYQTTEDALRDVLRLSK

SEQ ID NO: 104 - protein, 55 aa, Bacillus halodurans
ESLNGRFYTGTDMGTTMEDFVHALKETNGIVGIPKEYGGSGDSSVPTAKGVINSL

SEQ ID NO: 105 - protein, 57 aa, Bacillus halodurans
ATIARLKVKAIAGAANNQLEAERHGQMLHDQGIWFAPDYIVNSGGLIQVADELYGSN

SEQ ID NO: 106 - protein, 356 aa, Rhodococcus sp.
MSIDSALNWDGEMTVTRFDRETGAHFVIRLDSTQLGPAAGGTRAAQYSQLADALTDAGKLAGAMTLKMAVSNLPMGGGKSVIALPAPRHSIDPSTWARILRIHAENIDKLSGNYWTGPDVNTNSADMDTLNDTTEFVFGRSLERGGAGSSAFTTAVGVFEAMKATVAHRGLGSLDGLTVLVQGLGAVGGSLASLAAEAGAQLLVADTDTERVAHAVALGHTAVALEDVLSTPCDVFAPCAMGGVITTEVARTLDCSVVAGAANNVIADEAASDILHARGILYAPDFVANAGGAIHLVGREVLGWSESVVHERAVAIGDTLNQVFEISDNDGVTPDEAARTLAGRRAREASTTTATA

SEQ ID NO: 107 - protein, 4 aa, Rhodococcus sp.
NAGG

SEQ ID NO: 108 - protein, 33 aa, Rhodococcus sp.
LDSTQLGPAAGGTRAAQYSQLADALTDAGKLAG

SEQ ID NO: 109 - protein, 55 aa, Rhodococcus sp.
DKLSGNYWTGPDVNTNSADMDTLNDTTEFVFGRSLERGGAGSSAFTTAVGVFEAM

SEQ ID NO: 110 - protein, 58 aa, Rhodococcus sp.
EVARTLDCSVVAGAANNVIADEAASDILHARGILYAPDFVANAGGAIHLVGREVLGWS

SEQ ID NO: 111 - protein, 373 aa, Thermoactinomyces intermedius
MHHHHHHMRDVFEMMDRYGHEQVIFCRHPQTGLKAIIALHNTTAGPALGGCRMIPYASTDEALEDVLRLSKGMTYKCSLADVDFGGGKMVIIGDPKKDKSPELFRVIGRFVGGLNGRFYTGTDMGTNPEDFVHAARESKSFAGLPKSYGGKGDTSIPTALGVFHGMRATARFLWGTDQLKGRVVAIQGVGKVGERLLQLLVEVGAYCKIADIDSVRCEQLKEKYGDKVQLVDVNRIHKESCDIFSPCAKGGVVNDDTIDEFRCLAIVGSANNQLVEDRHGALLQKRSICYAPDYLVNAGGLIQVADELEGFHEERVLAKTEAIYDMVLDIFHRAKNENITTCEAADRIVMERLKKLTDIRRILLEDPRNSARR

SEQ ID NO: 112 - DNA, 1134 bp, Thermoactinomyces intermedius
catatgcatcaccatcaccaccacatgcgcgacgtgttcgaaatgatggatcgttatggccatgaacaggttatcttttgtcgccatccacaaacgggcttgaaagccattatcgccttacacaacacgacagcgggtccagctctgggtggttgccgtatgatcccgtatgcgtcaaccgatgaagcactcgaagatgttctgcgcctgtctaaaggtatgacctacaaatgctctctggcagatgtcgatttcggcggtggcaaaatggtcatcattggagatccgaagaaagacaaaagtccggaactgtttcgcgtcattggtcgcttcgtgggaggcttaaacggccggttttataccggtaccgatatggggactaaccctgaggactttgtgcatgcggcgcgtgaatcgaaaagctttgcaggactgcccaaatcctatggcggtaaaggcgatacctccatcccgacagccctgggcgtatttcatgggatgcgtgccactgcccgctttctctggggcacggatcagcttaaaggtcgggttgtcgcgattcaaggggttggcaaagttggcgaacgcctgttgcagttactcgtcgaggtaggcgcttactgcaagattgcggatatcgatagcgttcgttgtgagcaactgaaagagaaatacggggataaagtgcagttagtggacgtaaaccgcattcacaaggaaagctgcgatatcttttcgccttgtgctaaaggaggggtggtcaatgatgatacgattgacgagttccgctgcttggcgattgtgggctcagccaacaatcagctggtggaggatcgtcatggtgcgcttttgcagaaacgtagcatctgctatgcgccggactatctggtgaatgctggtggcctgatccaggttgcggacgaactggaaggtttccatgaagaacgtgtgctcgccaaaacggaagctatttacgacatggtactggacattttccatcgtgccaagaacgagaatatcaccacctgtgaagcagcagatcgcattgtgatggaacgcctgaagaaactgactgacattcggcgcattcttctggaagatccgcgcaatagtgcacgccgttaatgaggatcc

SEQ ID NO: 113 - DNA, 24 bp, Artificial Sequence
  linker
catatgcatcaccatcaccaccac

SEQ ID NO: 114 - DNA, 12 bp, Artificial Sequence
  linker
taatgaggatcc